(19) United States
(12) Patent Application Publication
(10) Pub. No.: US 2016/0058397 A1
(43) Pub. Date: Mar. 3, 2016

(54) MAGNETIC RESONANCE IMAGING (MRI) APPARATUS, METHOD OF CONTROLLING MRI APPARATUS, AND HEAD COIL FOR MRI APPARATUS

(71) Applicant: SAMSUNG ELECTRONICS CO., LTD., Suwon-si (KR)

(72) Inventors: Dae-hwan KIM, Suwon-si (KR); Jae-moon JO, Seongnam-si (KR)

(73) Assignee: SAMSUNG ELECTRONICS CO., LTD., Suwon-si (KR)

(21) Appl. No.: 14/747,294

(22) Filed: Jun. 23, 2015

(30) Foreign Application Priority Data
Aug. 29, 2014 (KR) 10-2014-0114519

(51) Int. Cl. A61B 5/00; A61B 5/055

(52) U.S. Cl. CPC A61B 5/7445; A61B 5/055

(57) ABSTRACT

Provided is a magnetic resonance imaging (MRI) apparatus including: a display configured to display a three-dimensional (3D) image on an inner wall of a bore within a gantry; a head coil comprising at least one opening formed at a region that corresponds to eyes of a target object and an optical element disposed in the at least one opening; and a controller configured to adjust a perspective of the 3D image based on an input received from the target object or an input received from a user.

### CROSS-REFERENCE TO RELATED APPLICATION

This application claims priority from Korean Patent Application No. 10-2014-0114519, filed on Aug. 29, 2014, in the Korean Intellectual Property Office, the disclosure of which is incorporated herein by reference in its entirety.

### BACKGROUND

1. Field

One or more exemplary embodiments relate to a magnetic resonance imaging (MRI) apparatus, a method for controlling the MRI apparatus, and a head coil for the MRI apparatus.

2. Description of the Related Art

An MRI apparatus uses a magnetic field to capture an image of a subject, and is widely used in the accurate diagnosis of diseases because it shows stereoscopic images of bones, lumbar discs, joints, and nerve ligaments at desired angles.

The MRI apparatus is configured to acquire MR signals and to reconstruct the acquired MR signals into an image to be output. In particular, the MRI apparatus acquires MR signals by using radio frequency (RF) coils, a permanent magnet, and gradient coils. To improve the performance of detection of the MR signals, an RF coil is used which is positioned close to an object and is attachable to and detachable from the object.

While an MRI apparatus captures an MR image of an object, the object must remain still within a bore of the MRI apparatus for a predetermined period of time. However, since the bore is a confined space and movement of the object is limited during MRI scanning, the object may feel confined and bored during the MRI scanning. In particular, patients having a fear of enclosed spaces (claustrophobia) or infants have difficulty in staying still within the bore for a predetermined period of time, and thus, MRI scanning may be performed only to a limited degree.

### SUMMARY

One or more exemplary embodiments include a magnetic resonance imaging (MRI) apparatus, a method for controlling the MRI apparatus, and a head coil for the MRI apparatus, which are used to relieve a feeling of confinement and boredom experienced by a target object during MRI scanning.

One or more exemplary embodiments include an MRI apparatus, a method for controlling the MRI apparatus, and a head coil for the MRI apparatus which allow a target object to conveniently view a three-dimensional (3D) image via a lens for viewing of the 3D image when the 3D image is provided within a bore of the MRI apparatus.

One or more exemplary embodiments include a head coil for use in MRI scanning, to or from which various types of lenses and filters are attachable or detachable.

Additional aspects will be set forth in part in the description which follows and, in part, will be apparent from the description, or may be learned by practice of the presented exemplary embodiments.

According to one or more exemplary embodiments, an MRI apparatus includes: a display configured to display a three-dimensional (3D) image on an inner wall of a bore within a gantry; a head coil comprising at least one opening formed at a region that corresponds to eyes of a target object and an optical element disposed in the at least one opening; and a controller configured to adjust a perspective of the 3D image based on at least one from among an input received from the target object and an input received from a user.

The 3D image may include a left-eye image polarized in a first direction and a right-eye image polarized in a second direction, and the optical element may include a left-eye polarizing filter configured to polarize light in the first direction and a right-eye polarizing filter configured to polarize light in the second direction.

The 3D image may be generated by combining a left-eye image represented by a first color component with a right-eye image represented by a second color component. The optical element may include a left-eye color filter configured to pass the first color component and a right-eye color filter configured to pass the second color component.

The optical element may be further configured to facilitate a reception of light by a first one of the eyes of the target object and to block light such that a second one of the eyes of the target object does not receive the blocked light, and the controller may be further configured to acquire a functional MRI (fMRI) image.

The head coil may further include at least one vision correction lens disposed in the at least one opening.

The 3D image may include a background image and a content image, and the controller may be further configured to change a perspective of the background image based on the at least one from among the input received from the target object and the input received from the user.

The 3D image may include at least one object that is focused behind the inner wall of the bore with respect to the target object.

The display may include at least one projector configured to project the 3D image onto the inner wall of the bore.

The at least one projector may be disposed inside the bore.

The MRI apparatus may further include a table on which the target object is placed and which is configured to enter and to exit the bore, and the at least one projector may be attached to the table and be disposed within the bore when the table is inside the bore.

The inner wall of the bore may have a printed background pattern, and the 3D image may include at least one object that is focused in front of the inner wall of the bore with respect to the target object.

The optical element may be attachable to the at least one opening and detachable from the at least one opening.

The optical element may include at least one from among a filter for viewing a 3D image, a light blocking filter, and a vision correction lens.

The head coil may further include a frame which includes an optical element holder that is formed around the at least one opening to have a surface step difference from an outer surface of the frame.

The frame of the head coil may further include a slot for providing a guide via which the optical element is inserted into the at least one opening.

The MRI apparatus may further include a table on which the target object is placed and which is configured to enter and to exit the bore, and the display may be further configured to change a position where the 3D image is displayed on the inner wall of the bore based on a distance traversed by the table while entering the bore.

The controller may be further configured to correct a distortion in the 3D image displayed on the inner wall of the bore.

According to one or more exemplary embodiments, a method for controlling an MRI apparatus includes: displaying a 3D image on an inner wall of a bore within a gantry of the MRI apparatus; and adjusting a perspective of the 3D image based on at least one from among an input received from a target object and an input received from a user, wherein the 3D image includes a left-eye image and a right-eye image. The left-eye image has at least one optical characteristic that corresponds to a left-eye optical filter disposed in an opening formed at a region in a head coil of the MRI apparatus that corresponds to a left eye of the target object, and the right-eye image has at least one optical characteristic that corresponds to a right-eye optical filter disposed in an opening formed at a region in the head coil of the MRI apparatus that corresponds to a right eye of the target object.

The 3D image may include a left-eye image polarized in a first direction and a right-eye image polarized in a second direction, and the left-eye optical filter may include a left-eye polarizing filter configured to polarize light in the first direction, and the right-eye optical filter may include a right-eye polarizing filter configured to polarize light in the second direction.

The 3D image may be generated by combining a left-eye image represented by a first color component with a right-eye image represented by a second color component. The left-eye optical filter may include a left-eye color filter configured to pass the first color component, and the right-eye optical filter may include a right-eye color filter configured to pass the second color component.

A first one of the left-eye optical filter and the right-eye optical filter may be configured to facilitate a reception of light by a corresponding one of the left eye of the target object and the right eye of the target object, and a second one of the left-eye optical filter and the right-eye optical filter may be configured to block light such that a corresponding one of the left eye of the target object and the right eye of the target object does not receive the blocked light, and the method may further include acquiring a fMRI image.

The 3D image may include a background image and a content image, and the method may further include changing a perspective of the background image based on the at least one from among the input received from the target object and the input received from the user.

The 3D image may include at least one object that is focused behind the inner wall of the bore with respect to the target object.

The method may further include projecting the 3D image onto the inner wall of the bore by using at least one projector.

The method may further include changing a position where the 3D image is displayed on the inner wall of the bore based on a distance traversed by a table while entering the bore.

The method may further include correcting a distortion in the 3D image displayed on the inner wall of the bore.

According to exemplary embodiments, during MRI scanning, a feeling of confinement and boredom experienced by a target object may be relieved.

Furthermore, a target object is able to conveniently view a 3D image via a lens for viewing of the 3D image when the 3D image is provided within a bore of the MRI apparatus.

In addition, any of various types of lenses and filters may be attachable to or detachable from a head coil for use in MRI scanning.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 illustrates a structure of a magnetic resonance imaging (MRI) apparatus, according to an exemplary embodiment;

FIG. 2 illustrates a structure of a head coil, according to an exemplary embodiment;

FIG. 3 illustrates a structure of a head coil, according to another exemplary embodiment;

FIG. 4 illustrates a structure of a head coil, according to another exemplary embodiment;

FIG. 5 is a flowchart of a method for controlling an MRI apparatus, according to an exemplary embodiment;

FIG. 6 illustrates a structure of a controller, according to an exemplary embodiment;

FIG. 7 is a diagram which illustrates an operation of adjusting a perspective of a three-dimensional (3D) image, according to an exemplary embodiment;

FIG. 8 is a diagram which illustrates a method for adjusting a perspective of a 3D image, according to an exemplary embodiment;

FIG. 9 is a diagram which illustrates a method for adjusting a perspective of a 3D image, according to another exemplary embodiment;

FIG. 10 is a diagram which illustrates a method for adjusting a perspective of a 3D image, according to another exemplary embodiment;

FIG. 11 is a diagram which illustrates a method for generating a 3D image, according to an exemplary embodiment;

FIG. 12 is a diagram which illustrates a method for adjusting a perspective of a 3D image, according to another exemplary embodiment;

FIG. 13 is a diagram which illustrates a method for adjusting a perspective of a 3D image, according to another exemplary embodiment;

FIG. 14 is a diagram which illustrates a displaying of a 3D image, according to the method of FIG. 13;

FIG. 15 illustrates an optical element, according to an exemplary embodiment;

FIG. 16 illustrates an optical element, according to another exemplary embodiment;

FIG. 17 illustrates a structure of a display, according to an exemplary embodiment;

FIG. 18 illustrates an optical element, according to another exemplary embodiment;

FIG. 19 illustrates a structure of a display, according to another exemplary embodiment;

FIG. 20 illustrates optical elements, according to an exemplary embodiment;

FIG. 21 is a flowchart of a method for controlling an MRI apparatus, according to an exemplary embodiment;

FIGS. 22A, 22B, and 22C illustrate a structure of a display, according to another exemplary embodiment;

FIG. 23 illustrates a light source driver of an in-bore projector, according to an exemplary embodiment;

FIG. 24 illustrates a structure of a light source driver and a light source unit in an in-bore projector, according to an exemplary embodiment;

FIG. 25 illustrates an inductor provided in an adjustable regulator and a bore, according to an exemplary embodiment;

FIG. 26 illustrates an example of driving signals that are applied to red, green, and blue light sources;

FIG. 27 illustrates a structure in which an optical element is attachable or detachable, according to an exemplary embodiment;

FIG. 28 illustrates a structure in which an optical element is attachable or detachable, according to another exemplary embodiment;

FIG. 29 illustrates a structure in which an optical element is attachable or detachable, according to another exemplary embodiment; and

FIG. 30 illustrates a structure of an MRI system, according to an exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present inventive concept. As used herein, the term “and/or” includes any and all combinations of one or more of the associated listed items. Expressions such as “at least one of,” when preceding a list of elements, modify the entire list of elements and do not modify the individual elements of the list.

Advantages and features of one or more exemplary embodiments and methods and apparatuses of accomplishing the same may be understood more readily by reference to the following detailed description of the exemplary embodiments and the accompanying drawings. In this regard, the present exemplary embodiments may have different forms and should not be construed as being limited to the descriptions set forth herein. Rather, these exemplary embodiments are provided so that this disclosure will be thorough and complete and will fully convey the concept of the present exemplary embodiments to one of ordinary skill in the art, and the present inventive concept will only be defined by the appended claims.

Hereinafter, the terms used in the specification will be briefly described, and then the exemplary embodiments will be described in detail.

The terms used in this specification are those general terms currently widely used in the art in consideration of functions regarding the exemplary embodiments, but the terms may vary according to the intention of those of ordinary skill in the art, precedents, or new technology in the art. Further, some terms may be arbitrarily selected by the applicant, and in this case, the meaning of the selected terms will be described in detail in the detailed description of the exemplary embodiments. Thus, the terms used herein have to be defined based on the meaning of the terms together with the description throughout the specification.

When a part “includes” or “comprises” an element, unless there is a particular description contrary thereto, the part can further include other elements, not excluding the other elements. Further, the term “unit” in the exemplary embodiments means a software component or hardware component such as a field-programmable gate array (FPGA) or an application-specific integrated circuit (ASIC), and performs a specific function. However, the term “unit” is not limited to software or hardware. The “unit” may be formed so as to be in an addressable storage medium, or may be formed so as to operate one or more processors. Thus, for example, the term “unit” may refer to components such as software components, object-oriented software components, class components, and task components, and may include processes, functions, attributes, procedures, subroutines, segments of program code, drivers, firmware, micro codes, circuits, data, a database, data structures, tables, arrays, or variables. A function provided by the components and “units” may be associated with the smaller number of components and “units”, or may be divided into additional components and “units”.

Reference will now be made in detail to exemplary embodiments, examples of which are illustrated in the accompanying drawings. In the following description, well-known functions or constructions are not described in detail so as not to obscure the exemplary embodiments with unnecessary detail.

Throughout the specification, an “image” may mean multi-dimensional data formed of discrete image elements, e.g., pixels in a two-dimensional (2D) image and voxels in a three-dimensional (3D) image. For example, an image may include a medical image of an object acquired by any of an X-ray apparatus, a computed tomography (CT) apparatus, a magnetic resonance imaging (MRI) system, an ultrasound diagnosis apparatus, or another medical imaging apparatus.

Furthermore, in the present specification, an “object” may be a human, an animal, or a part of a human or animal. For example, the object may be an organ (e.g., the liver, the heart, the womb, the brain, a breast, or the abdomen), a blood vessel, or a combination thereof. The object may be a phantom. The phantom means a material having a density, an effective atomic number, and a volume that are approximately the same as those of an organism. For example, the phantom may be a spherical phantom having properties similar to the physical body.

Throughout the specification, a “user” may be, but is not limited to, a medical expert, for example, a medical doctor, a nurse, a medical laboratory technologist, or a medical imaging expert, or a technician who repairs medical apparatuses.

Furthermore, in the present specification, an “MR image” refers to an image of an object obtained by using the nuclear magnetic resonance principle.

Furthermore, in the present specification, a “pulse sequence” refers to continuity of signals repeatedly applied by an MRI apparatus. Furthermore, in the present specification, a “pulse sequence” refers to continuity of signals repeatedly applied by an MRI apparatus.

An MRI system is an apparatus configured for acquiring a sectional image of a part of an object by expressing, in a contrast comparison, a strength of a MR signal with respect to a radio frequency (RF) signal generated in a magnetic field having a specific strength. For example, if an RF signal that only resonates a specific atomic nucleus (for example, a hydrogen atomic nucleus) is emitted for an instant toward the object placed in a strong magnetic field and then such emission stops, an MR signal is emitted from the specific atomic nucleus, and thus the MRI system may receive the MR signal and acquire an MR image. The MR signal denotes an RF signal emitted from the object. An intensity of the MR signal may be determined according to any of a density of a predetermined atom (for example, hydrogen) of the object, a relaxation time T1, a relaxation time T2, and a flow of blood and/or the like.

MRI systems include characteristics that are different from those of other imaging apparatuses. Unlike imaging apparatuses such as CT apparatuses that acquire images according to a direction of detection hardware, MRI systems may acquire 2D images or 3D volume images that are oriented toward an optional point. MRI systems do not expose objects or examiners to radiation, unlike CT apparatuses, X-ray apparatuses, position emission tomography (PET) apparatuses, and single photon emission CT (SPECT) apparatuses, may acquire images having high soft tissue contrast, and may acquire neurological images, intravascular images, musculoskeletal images, and oncologic images that are required to precisely capturing abnormal tissues.

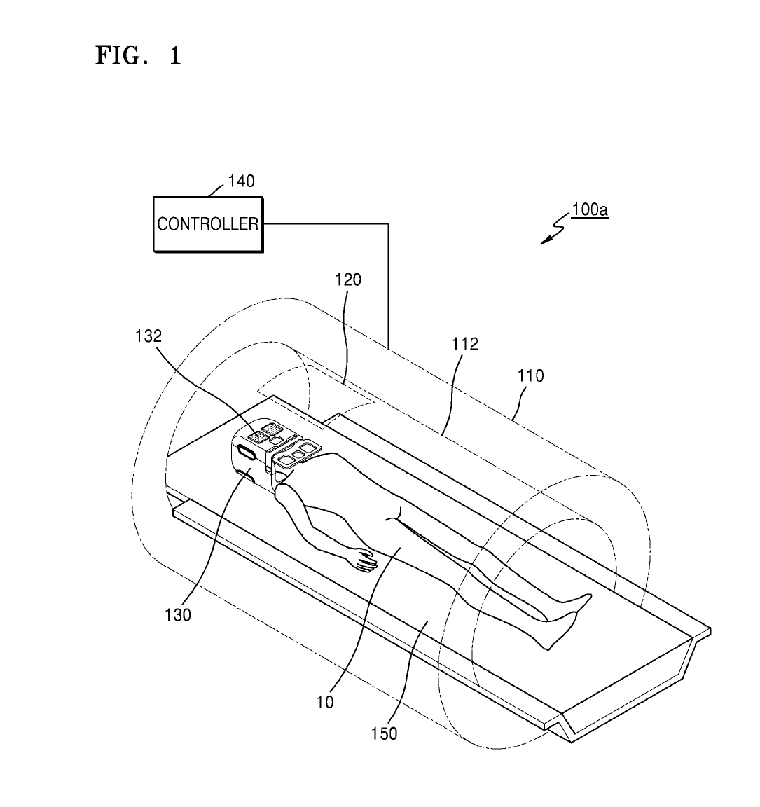

FIG. 1 illustrates a structure of an MRI apparatus 100a, according to an exemplary embodiment. The MRI apparatus 100a according to the present exemplary embodiment includes a gantry 110, a display 120, a head coil (also referred to herein as a “headgear”) 130, and a controller 140.

The gantry 110 produces a magnetic field therein and blocks electromagnetic waves from being externally emitted. For example, the gantry 110 may be formed in a cylindrical shape and have a bore formed therein. A magnetostatic field and a gradient magnetic field are formed at the bore in the gantry 110, and an RF signal is irradiated towards a target object 10. During an MRI session, the target object 10 lies on a table 150 that then moves into the bore of the gantry 110, and undergoes the MRI for a predetermined period of time during which the target object 10 stays in the bore.

The gantry 110 may have a main magnet, a gradient coil, an RF coil, etc. stacked together. The gantry 110 accommodates the main magnet and the gradient coil, which are configured to generate a magnetostatic field and a gradient field, respectively, and the RF coil, which is configured to irradiate an RF signal. The RF coil may irradiate an RF signal toward a patient and receive an MR signal emitted from the patient. In detail, the RF coil may transmit an RF signal at a same frequency as precessional motion to the patient towards atomic nuclei in precessional motion, cease a transmission of the RF signal, and then receive an MR signal emitted from the patient.

For example, in order to cause an atomic nucleus to transition from a low energy state to a high energy state, the RF coil may generate and apply an electromagnetic wave signal that is an RF signal corresponding to a type of the atomic nucleus, to the target object 10. When the electromagnetic wave signal generated by the RF coil is applied to the atomic nucleus, the atomic nucleus may transit from the low energy state to the high energy state. Then, when electromagnetic waves generated by the RF coil disappear, the atomic nucleus to which the electromagnetic waves were applied transits from the high energy state to the low energy state, thereby emitting electromagnetic waves having a Larmor frequency. In this aspect, when the applying of the electromagnetic wave signal to the atomic nucleus is ceased, an energy level of the atomic nucleus is changed from a high energy level to a low energy level, and thus the atomic nucleus may emit electromagnetic waves having a Larmor frequency. The RF coil may receive electromagnetic wave signals from atomic nuclei included in the target object 10.

The RF coil may be fixed to the gantry 110 or may be detachable. When the RF coil is detachable, the RF coil 26 may include an RF coil that is designed for a part of the object, such as any of a head RF coil, a chest RF coil, a leg RF coil, a neck RF coil, a shoulder RF coil, a wrist RF coil, and/or an ankle RF coil.

The head coil 130 is shaped to surround a head of the target object 10, as shown in FIG. 10. The head coil 130 may have one side that opens so that it is attachable to and detachable from the target object 10. The head coil 130 may have openings 132 at regions that correspond to eyes of the target object 10, which enables the target object 10 to see outside of the head coil 130 even when wearing the head coil 130. The head coil 130 may also include an optical element in the openings 132.

The MRI apparatus 100a includes a display 120 for displaying a 3D image on an inner wall 112 of the bore. For example, the display 120 may be implemented as a projection type display. As another example, the display 120 may be implemented as any of a liquid crystal display (LCD) panel, an organic light-emitting display panel, etc. formed of a non-metallic material.

A 3D image includes at least one object which is depicted at different focal distances, and thus, gives a stereoscopic effect. The 3D image may be represented using any of polarization, anaglyph, and other methods.

According to the present exemplary embodiment, an optical filter for viewing a 3D image may be disposed in the opening 132 of the head coil 130 so that the target object 10 may view a 3D image displayed on the display 120. Thus, the target object 10 is able to conveniently see a 3D image without wearing any separate glasses.

The controller 140 controls overall operations of the MRI apparatus 100a. The controller 140 may adjust a perspective of the 3D image based on an input received from the target object 10 and/or an input received from a user such as a medical practitioner.

Adjusting a perspective of a 3D image means changing a position at which at least one object in the 3D image is focused. For example, the controller 140 may adjust the perspective of a 3D image by making letters contained in the 3D image appear closer to or farther away from the object 10 than they are currently displayed, based on an input received from either the target object 10 or a user.

The input from the target object 10 may be provided via any of a terminal that may be held by the target object 10, a user input element disposed on the table 150, a user input element disposed on the inner wall 112 of the bore, etc. For example, the user input elements may include any one or more of buttons, keys, a pressure sensor, a touch screen, a touch sensor, dials, and/or the like.

For example, the input from the user such as a medical practitioner may be provided via any of a user input element provided in an operating unit of the MRI apparatus 100a, a user input element provided on an outer wall of the gantry 112, a terminal that may be held by the user, etc. For example, the user input elements may include any one or more of buttons, keys, a pressure sensor, a touch screen, a touch sensor, dials, or the like.

The controller 140 may also perform various operations such as controls of the gantry 110, the table 150, and the display 120, monitoring of the MR apparatus 100a and the target object 10, generation and outputting of a 3D image, and/or processing and storage of an MR image, etc.

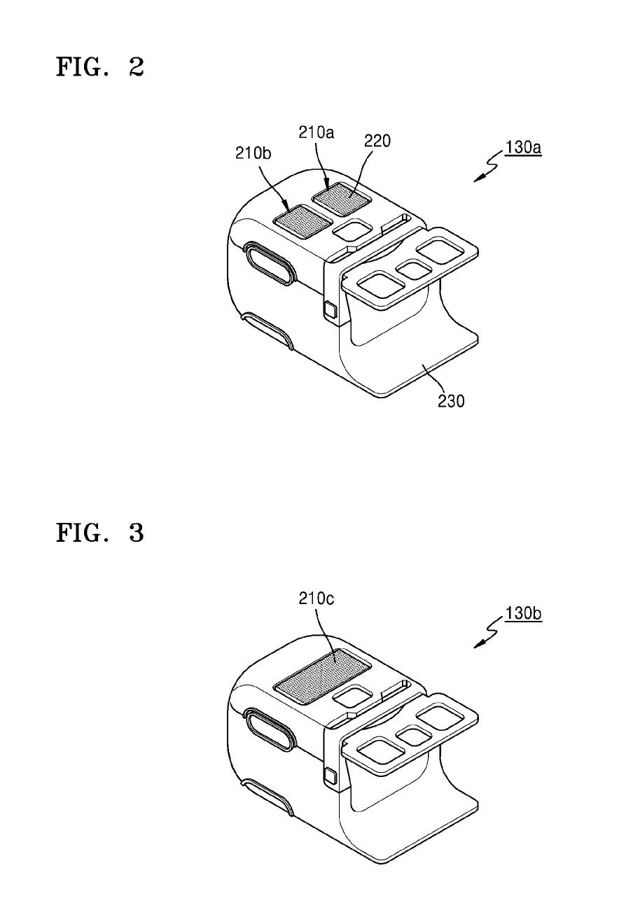

FIG. 2 illustrates a structure of a head coil 130a, according to an exemplary embodiment.

The head coil 130a has a frame 230 that is shaped to surround a head of the target object (10 of FIG. 1) and includes a plurality of RF coils therein. The RF coils are arranged in a region of the frame 230 where openings 210a and 210b are not formed, or to surround the openings 210a and 210b.

The head coil 130a includes the openings 210a and 210b that are formed at regions respectively corresponding to a left eye and a right eye of the target object 10, and optical elements 220 that are disposed on the openings 210a and 210b.

According to an exemplary embodiment, the optical elements 220 include optical filters for viewing a 3D image, such as a polarizing filter or color filter. Filters for left-eye and right-eye images may be disposed in the openings 210a and 210b, respectively.

According to another exemplary embodiment, the optical elements 220 may include a light blocking filter. For example, a light blocking filter may be disposed in the opening 210a corresponding to the left eye, but it may not be disposed in the opening 210b corresponding to the right eye. Conversely, a light blocking filter may not be disposed in the opening 210a but may be disposed in the opening 210b. In the present exemplary embodiment, the light blocking filter may be used when MRI scanning is performed with one eye of the target object 10 closed in order to acquire a functional MRI (fMRI) image.

According to another exemplary embodiment, the optical elements 220 may include vision correction lenses. In the present exemplary embodiment, vision correction lenses may be disposed in the openings 210a and 210b, respectively, according to vision of the target object 10.

In an exemplary embodiment, the optical elements 220 may be disposed in the openings 210a and 210b, respectively, in an attachable and detachable manner.

FIG. 3 illustrates a structure of a head coil 130b, according to another exemplary embodiment.

Referring to FIG. 3, the head coil 130b according to the present exemplary embodiment includes an opening 210c formed at a region corresponding to left and right eyes of the target object (10 of FIG. 1). According to the present exemplary embodiment, the optical element (220 of FIG. 2) may have optical elements for left and right eyes integrally formed together and may be disposed in the opening 210c. Due to the absence of a frame member between the left eye and the right eye, it is possible to provide a an expansive view for the target object 10 and further reduce feelings of confinement that the target object 10 may experience.

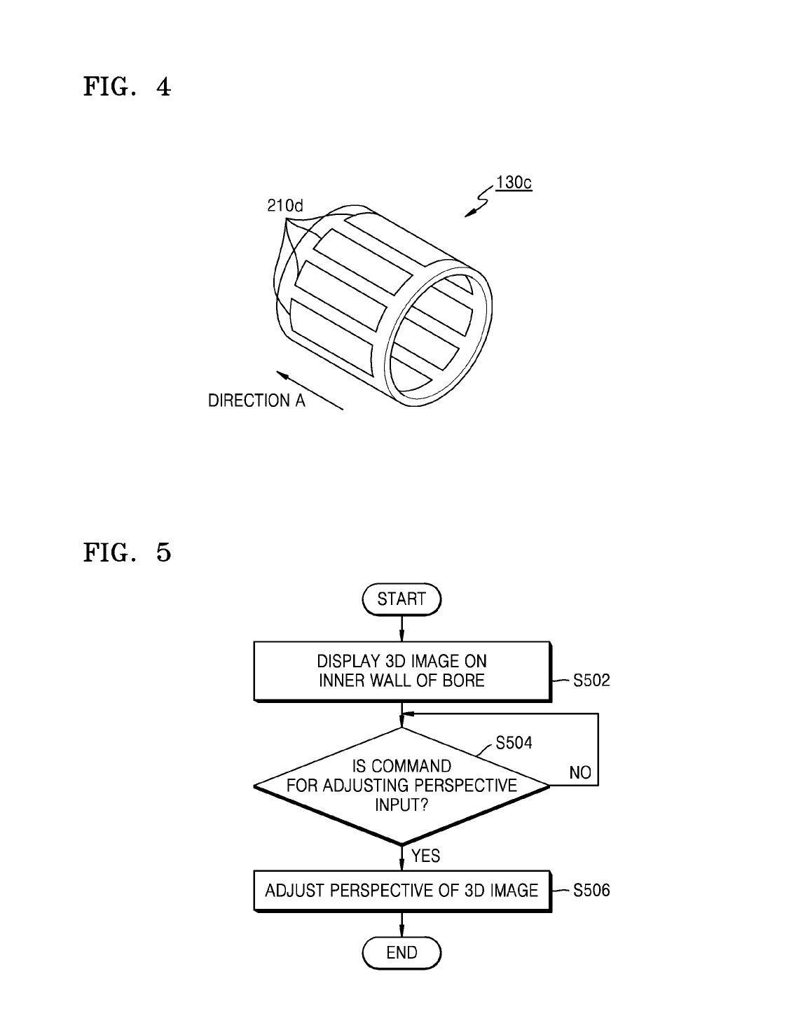

FIG. 4 illustrates a structure of a head coil 130c, according to another exemplary embodiment.

Referring to FIG. 4, the head coil 130c according to the present exemplary embodiment may include a plurality of openings 210d that extend along a direction A in which a head of the target object 10 may be inserted into the head coil 130c. According to the present exemplary embodiment, a user may select two of the openings 210d formed at positions that correspond to the left eye and the right eye of the target object 10 and place the optical elements 220 in the selected two openings 210d.

FIG. 5 is a flowchart of a method for controlling an MRI apparatus, according to an exemplary embodiment.

The method according to the present exemplary embodiment may be performed by the MRI apparatus 100a of FIG. 1. However, the method may be performed by any of various MRI apparatuses without departing from the spirit and scope of the present inventive concept. For a purpose of describing the present exemplary embodiment, it is assumed that the method is performed by the MRI apparatus 100a of FIG. 1.

Referring to FIGS. 1 and 5, in operation S502, the MRI apparatus 100a displays a 3D image on an inner wall of the bore. For example, as shown in FIG. 1, the 3D image may be displayed on the display 120 disposed on the inner wall of the bore.

Next, in operation S504, when a command for adjusting a perspective of the 3D image is received from the target object 10 or from a user, in operation S506, the MRI apparatus 100a adjusts the perspective of the 3D image. As described above, the perspective of the 3D image may be adjusted by controlling a position at which at least one object in the 3D image is focused.

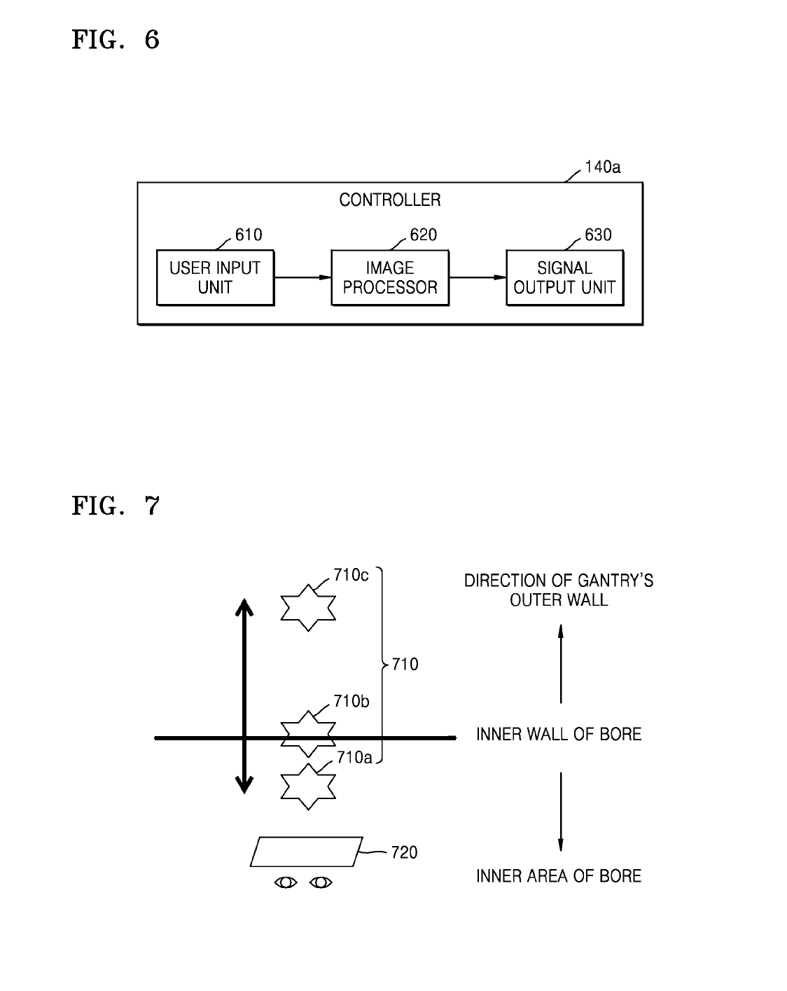

FIG. 6 illustrates a structure of a controller 140a, according to an exemplary embodiment.

Referring to FIGS. 1 and 6, the controller 140a according to the present exemplary embodiment includes a user input unit (also referred to herein as a “user input device”) 610, an image processor 620, and a signal output unit (also referred to herein as a “signal output device”) 630.

The user input unit 610 receives an input from the target object 10 and/or from a user. For example, the user input unit 610 may include a portable terminal and user input elements disposed on a table, an inner wall of a bore, and an outer wall of the gantry 110 and in an operating unit of the MRI apparatus 100a.

Furthermore, for example, the user input unit 610 may include any one or more of buttons, keys, a pressure sensor, a touch screen, a touch sensor, dials, and/or the like.

The image processor 620 adjusts a perspective of a 3D image based on an input received from at least one of a target object and/or a user via the user input unit 610. The image processor 620 also outputs the 3D image for which a perspective has been adjusted to the signal output unit 630.

The signal output unit 630 outputs a signal corresponding to the 3D image to the display 120. For example, the signal output unit 630 may perform operations such as amplification of an output signal, removal of noise, and emulation. The signal output unit 630 may also perform operations such as adjustment of timing when a left-eye image signal and a right-eye image signal are output to the display 120, selection of an output path, etc.

FIG. 7 is a diagram which illustrates an operation of adjusting a perspective of a 3D image, according to an exemplary embodiment. Referring to FIGS. 1 and 7, the controller 140 may adjust a position at which an object 710 in the 3D image is focused according to an input received from the target object 10 and/or an input received from a user. For example, in order to display an object 710a, the controller 140 may move a position of an object 710b that is focused on an inner wall of a bore toward an inner area of the bore, i.e., toward the target object 10 according to an input received from the target object 10 and/or an input received from the user. Furthermore, in order to display an object 710c, the controller 140 may move the position of the object 710b that is focused on the inner wall of the bore towards an outer wall of the gantry 110, i.e., away from the target object 10, according to an input received from either of the target object 10 or the user.

The target object 10 may view the 3D image via an optical filter 720 disposed in the head coil 130, 130a, 130b, or 130c.

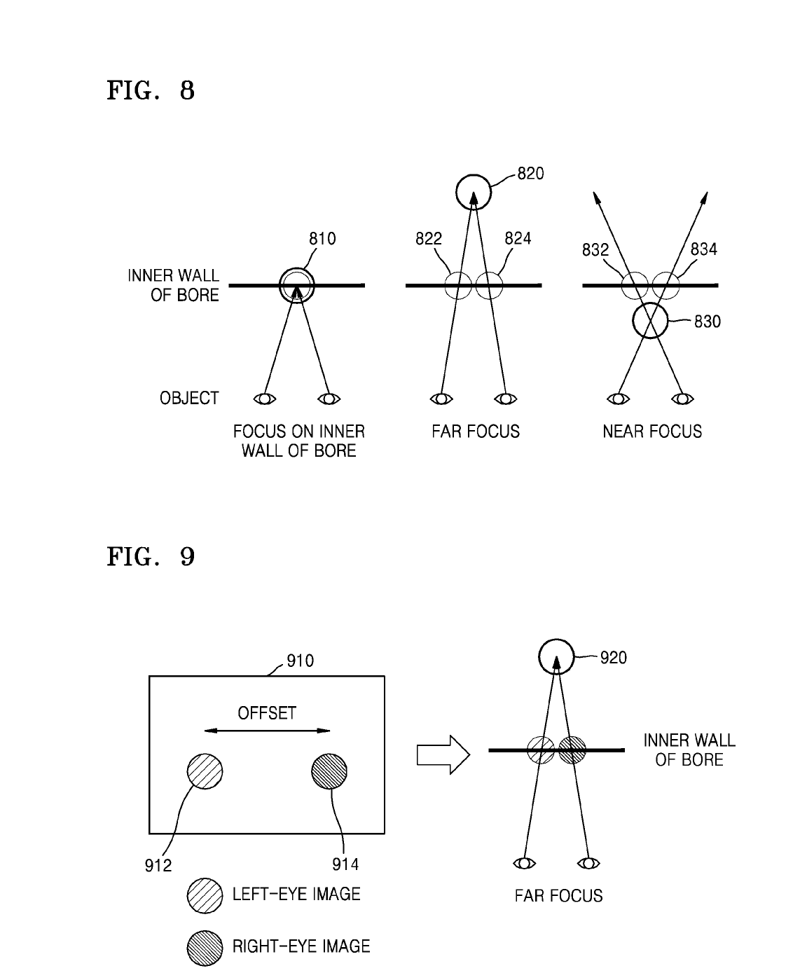

FIG. 8 is a diagram which illustrates a method for adjusting a perspective of a 3D image, according to an exemplary embodiment.

The perspective of the 3D image is adjusted by controlling a position at which an object in the 3D image is focused. For example, objects 810, 820, and 830 in the 3D image may be focused on, behind, and in front of an inner wall of a bore, respectively, and displayed.

If focusing is performed behind the inner wall of the bore, objects in a left-eye image and a right-eye image are displayed at positions 822 and 824, respectively. In this case, when seeing the objects in the left- and right-eye image respectively displayed at the positions 822 and 824, the target object 10 perceives the objects as being at the same position as the object 820.

If focusing is performed in front of the inner wall of the bore, objects in a left-eye image and a right-eye image are displayed at positions 834 and 832, respectively. In this case, when seeing the objects in the left- and right-eye image respectively displayed at the positions 834 and 832, the target object 10 perceives the objects as being at the same position as the object 830.

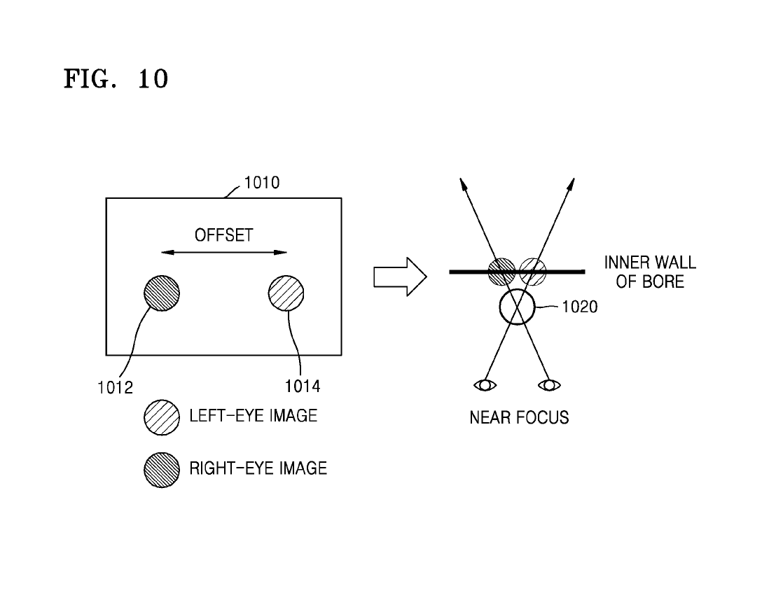

FIG. 9 is a diagram which illustrates a method for adjusting a perspective of a 3D image, according to another exemplary embodiment.

Referring to FIGS. 1 and 9, when an object 920 in a 3D image 910 is to be focused behind an inner wall of the bore, an object 912 in a left-eye image is placed on the left side of the 3D image 910 being displayed, while an object 914 in a right-eye image is placed on the right side thereof. An offset that is a distance between the objects 912 and 914 in the left-eye and right-eye images may be adjusted, thereby controlling a perspective of the 3D image 910. In this case, as the offset increases, the object 920 appears to be farther away from the target object 10, i.e., the object 920 recedes behind the inner wall of the bore. Conversely, as the offset decreases, the object 920 appears to be closer to the target object 10, i.e., the object 920 moves towards an inner area of the bore.

FIG. 10 is a diagram which illustrates a method for adjusting a perspective of a 3D image, according to another exemplary embodiment.

When an object 1020 in a 3D image 1010 is to be focused within the bore (i.e., in front of an inner wall of the bore), an object 1014 in a left-eye image is placed on the right side of the 3D image 1010 being displayed, while an object 1012 in a right-eye image is placed on the left side thereof. An offset that is a distance between the objects 1014 and 1012 in the left-eye and right-eye images may be adjusted, thereby controlling a perspective of the 3D image 1010. In this case, as the offset increases, the object 1020 appears to be closer to the target object 10, i.e., the object 1020 approaches an inner area of the bore. Conversely, as the offset decreases, the object 1020 appears to be farther away from the target object 10, i.e., the object 1020 moves towards an inner wall of the bore.

When the offset is zero (i.e., 0), the object 1020 in the 3D image 1010 is focused on the inner wall of the bore, and thus, appears to be on the inner wall of the bore.

As described above, the controller 140 may control a prospective of a 3D image by adjusting positions of objects in left-eye and right-eye images and an offset between the objects.

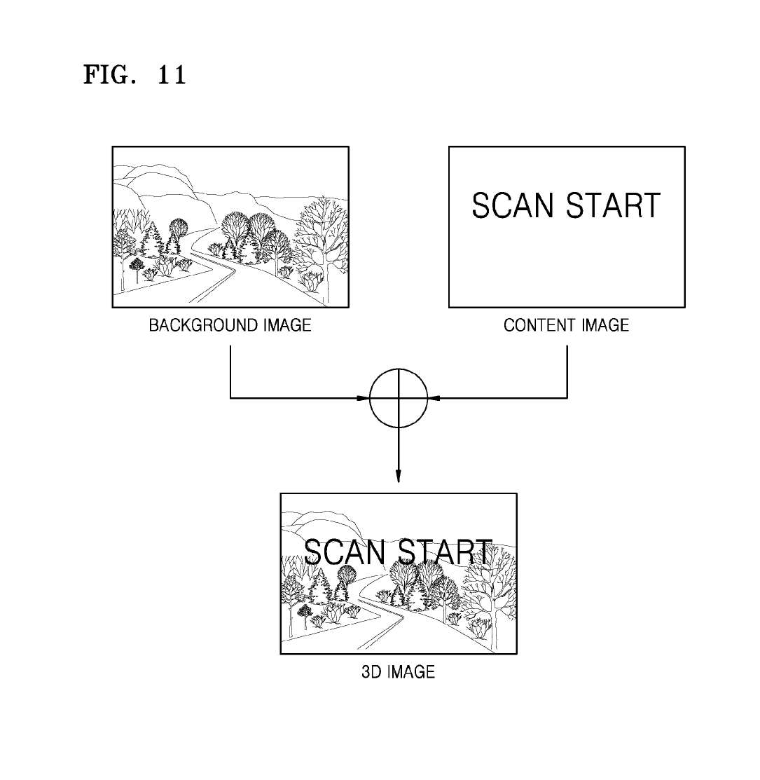

FIG. 11 is a diagram which illustrates a method for generating a 3D image, according to an exemplary embodiment.

According to the present exemplary embodiment, the 3D image is generated by combining a background image with a content image. The background image may be captured by a camera, and the content image may have a text included therein. Alternatively, the background image and the content image may include different objects. For example, the background image may include an image of the night sky, and the content image may include an image of the moon and stars.

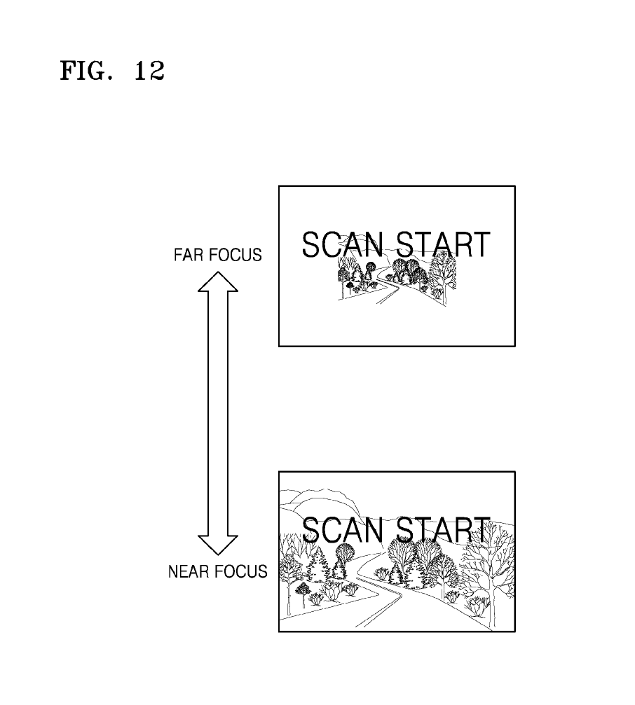

FIG. 12 is a diagram which illustrates a method for adjusting a perspective of a 3D image, according to another exemplary embodiment.

Referring to FIGS. 1 and 12, according to the present exemplary embodiment, the controller 140 may adjust a perspective of the 3D image by controlling a perspective of a background image. For example, as shown in FIG. 12, the controller 140 may move a position where a background image is focused away from and towards the target object 10, thereby generating far-focused and near-focused images, respectively.

According to the present exemplary embodiment, the target object 10 perceives the background image as being distant therefrom and an inner area of the bore as being wider than it actually is. Thus, it is possible to relieve feelings of confinement that the target object 10 may experience while staying in the bore.

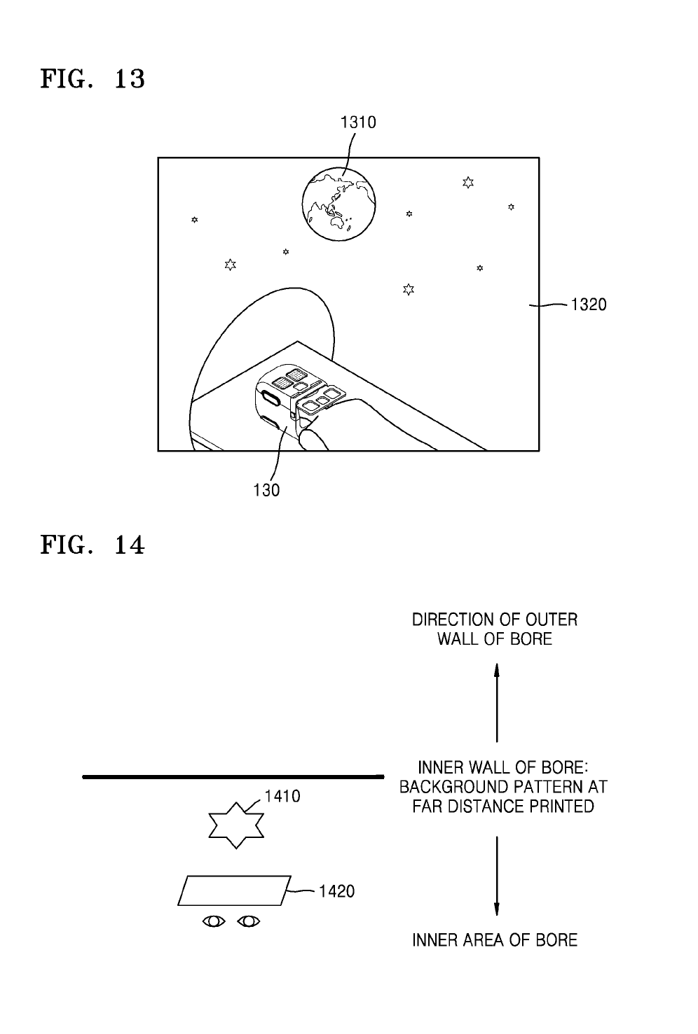

FIG. 13 is a diagram which illustrates a method for adjusting a perspective of a 3D image 1310, according to another exemplary embodiment.

According to the present exemplary embodiment, a background pattern 1320 is printed on an inner wall, and the 3D image 1310 may be displayed on the background pattern 1320. For example, the background pattern and the 3D image 1310 may represent the night sky and the Earth, respectively.

FIG. 14 is a diagram which illustrates a displaying of a 3D image according to the method of FIG. 13.

Referring to FIGS. 1, 13, and 14, in the present exemplary embodiment, the controller 140 may place a position where an object 1410 (e.g., the Earth as shown in FIG. 13) in the 3D image is focused in front of an inner wall of a bore, i.e., within the bore. According to the present exemplary embodiment, when seeing the 3D image via an optical filter 1420 for viewing a 3D image, the target object 10 perceives the inner wall of the bore on which a background pattern is printed as being at a relatively far distance due to perception of the object 1410 as being at a relatively near distance. Thus, according to the present exemplary embodiment, the target object 10 perceives an inner area of the bore as being wider than it actually is, and thus, a feeling of confinement may be mitigated.

According to an exemplary embodiment, the controller 140 may correct a distortion in an image displayed on the inner wall 112 of the bore. The inner wall 112 of the bore is curved, and thus, the bore has a cylindrical cross-section. Thus, an image projected onto the inner wall 112 of the bore may undergo a curved surface distortion, due to the curved shape of the inner wall 112 of the bore. Furthermore, when viewed from a longitudinal section of the gantry 110, a light beam may be projected from one side obliquely with respect to the inner wall 112 of the gantry 110. The obliqueness of the projection may cause a skew distortion. When a direction in which a projector of the display 120 projects an image moves along the inner wall 112 of the bore, an image projected on the inner wall 112 of the bore suffers from a curved surface distortion, due to the curved shape of the inner wall 112 of the bore. When a direction in which the projector of the display 120 projects an image moves in a longitudinal direction of the inner wall 112 of the bore, the amount of skew distortion may be changed. As the direction in which the projector projects an image changes, the controller 140 may remove a curved surface distortion in an image formed on the curved inner wall 112 of the bore by generating in advance a preceding primary distortion that offsets or cancels out a curved surface distortion and/or a skew distortion during image signal processing.

According to an exemplary embodiment, the MRI apparatus 100a may vary a position where a 3D image is displayed on the inner wall 112 of the bore based on where the table 150 enters the bore. For example, the display 120 may include the projector for projecting an image onto the inner wall 112 of the bore, and a position on the inner wall 112 of the bore where an image scanned by the projector is formed may vary based on a position to which the table 150 enters the bore.

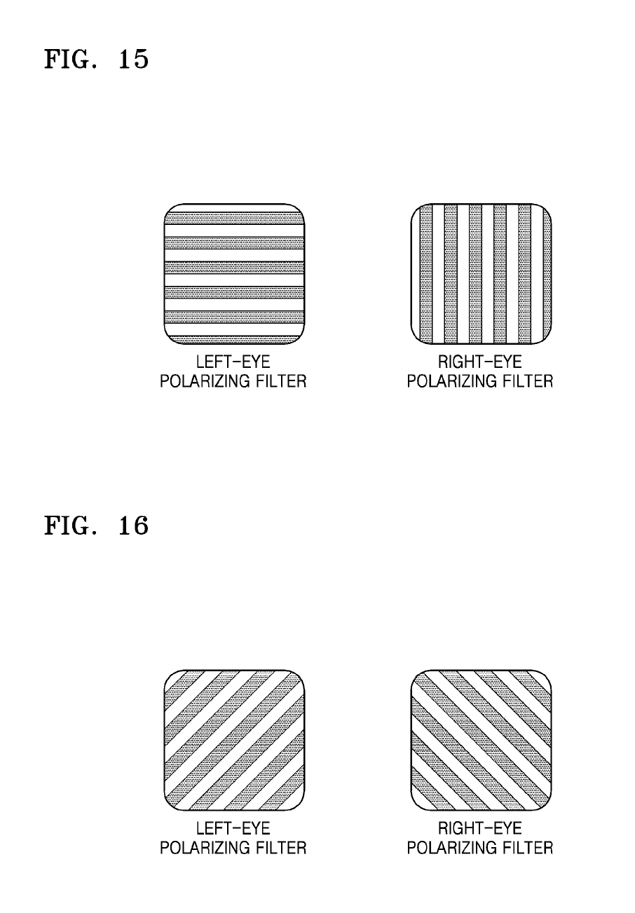

FIG. 15 illustrates an optical element, according to an exemplary embodiment.

Referring to FIG. 15, according to an exemplary embodiment, the optical element includes a left-eye polarizing filter that polarizes light in a transverse direction and a right-eye polarizing filter that polarizes light in a longitudinal direction. For example, the left-eye and right-eye polarizing filters may be disposed in the openings (210a and 210b of FIG. 2) of the head coil (130a of FIG. 2) that respectively correspond to the left eye and the right eye of the target object 10.

FIG. 16 illustrates an optical element, according to another exemplary embodiment.

In an exemplary embodiment, the optical element includes a left-eye polarizing filter configured for polarizing light at 45 degrees to the right of vertical and a right-eye polarizing filter configured for polarizing light at 45 degrees to the left of vertical. For example, the left-eye and right-eye polarizing filters may be disposed in the openings (210a and 210b of FIG. 2) of the head coil (130a of FIG. 2) that respectively correspond to the left eye and the right eye of the target object 10.

When left-eye and right-eye images of a 3D image are generated by using a polarization technique, for example, the optical element may be implemented as a polarizing filter as shown in FIGS. 15 and 16. In this case, polarization patterns of left-eye and right-eye polarizing filters correspond to polarization patterns of left-eye and right-eye images, respectively.

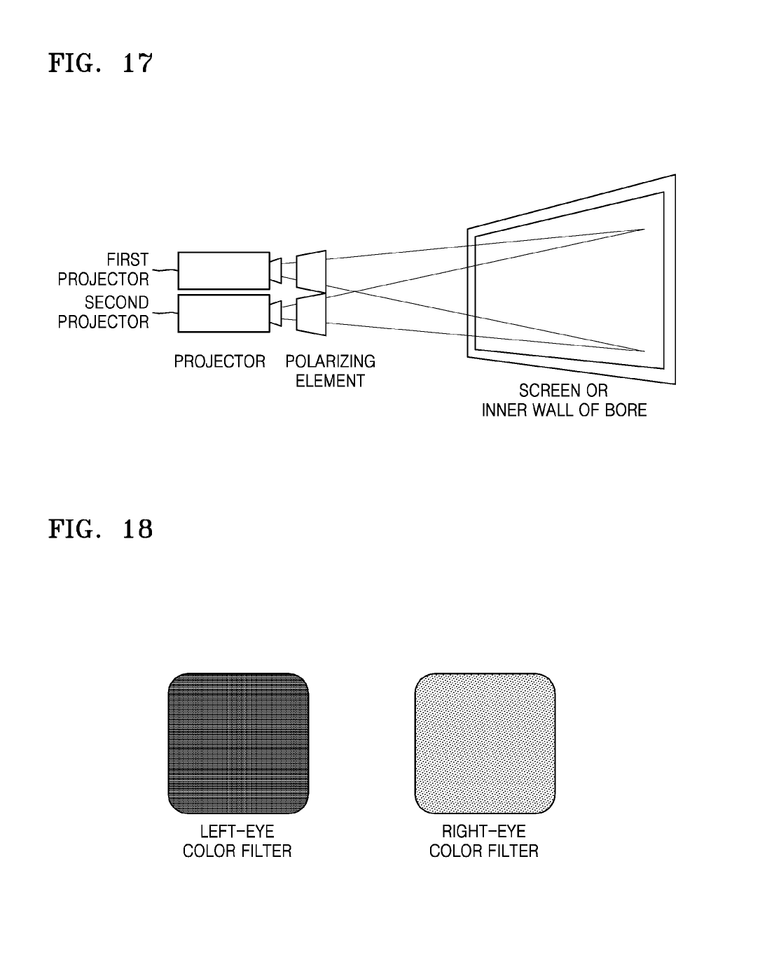

FIG. 17 illustrates a structure of the display 120 of FIG. 1, according to an exemplary embodiment.

The display 120 according to the present exemplary embodiment includes a first projector, a second projector, and a screen. According to an exemplary embodiment, the screen may be disposed on a portion of an inner wall of a bore and be made of a material (e.g., silver (Ag)) that has a relatively high light reflectivity. In another exemplary embodiment, the projector may project light directly onto the inner wall of the bore without employing a separate screen.

Each of the first projector and the second projector may include a respective polarizing element via which light is emitted from the first and second projectors. Polarization patterns of the polarizing elements correspond to respective polarization patterns of left-eye and right-eye polarizing filters. For example, if the first and second projectors may project left-eye and right-eye images, respectively, the polarizing elements of the first and second projectors may polarize light in the same respective patterns as the polarization patterns of the left-eye and right-eye polarizing filters.

For example, the first projector and the second projector may be mounted at portions of the table (150 of FIG. 1) that do not enter the gantry (110 of FIG. 1) or on a predetermined holder that is outside of the gantry 110. The first and second projectors may be disposed to project light on a region that corresponds to the display 120 on the inner wall (112 of FIG. 1).

FIG. 18 illustrates an optical element, according to another exemplary embodiment.

According to an exemplary embodiment, the optical element may include a color filter that allows only predetermined color components to pass therethrough. In this case, a left-eye color filter may pass a first color component while a right-eye color filter may pass a second color component. For example, the left-eye and right-eye color filters may be disposed in the openings (210a and 210b of FIG. 2) of the head coil (130a of FIG. 2) that respectively correspond to the left eye and the right eye of the target object 10.

In an exemplary embodiment, if a 3D image is generated by using an anaglyph method, left-eye and right-eye images may be represented as images that are respectively tinted in first and second color components. For example, the first and second color components may be red-blue and red-green, respectively. In this case, the left-eye and right-eye color filters pass the first and second color components, respectively, and when seeing the 3D image via the left-eye and right-eye color filters, the target object 10 may feel a sense of depth provided by the 3D image.

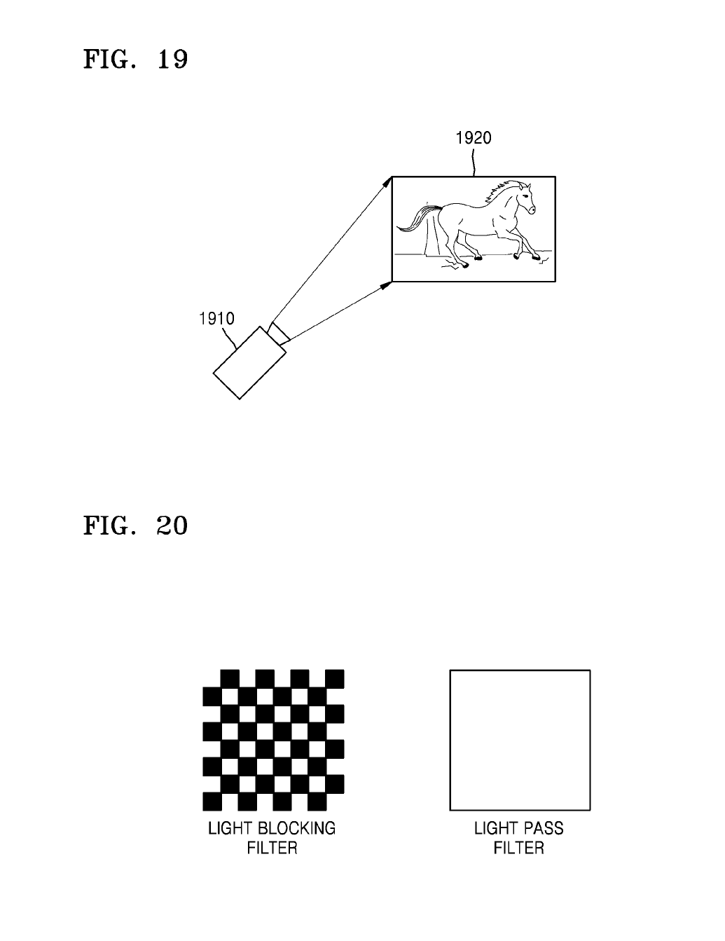

FIG. 19 illustrates a structure of the display (120 of FIG. 1), according to another exemplary embodiment.

Referring to FIGS. 1 and 19, according to the present exemplary embodiment, a 3D image 1920 may be displayed by using an anaglyph method that utilizes a single projector 1910. For example, an anaglyph-based conversion of a 3D image may be performed by the controller 140 or the projector 1910. As another example, a 3D image may be stored by using an anaglyph method. According to the present exemplary embodiment, the head coil 130 may include left-eye and right-eye color filters as shown in FIG. 18.

Various methods other than the anaglyph method may be used to display the 3D image 1920 using the single projector 1910.

According to an exemplary embodiment, a 3D image may be displayed using the single projector 1910 by placing a device for alternately changing between left-eye and right-eye polarizing filters in a path along which light is output from the projector 1910.

According to another exemplary embodiment, the projector 1910 may include two light sources that may respectively display left-eye and right-eye images.

In another exemplary embodiment, the projector 1910 uses a shutter glass-based method and may be synchronized with shutter glasses worn by the target object 10 in order to output left-eye and right-eye images.

According to an exemplary embodiment, the target object 10 may view a 3D image while wearing both 3D glasses and the head coil 130. For example, the target object 10 may wear any of glasses equipped with polarizing filters, shutter glasses, and/or glasses with color filters according to a method whereby the display 120 displays a 3D image. 3D glasses may be formed of a non-metallic material such as plastic. When the target object 10 wears 3D glasses, the head coil 130 may include different types of optical elements (e.g., a vision correction lens, a light blocking filter, etc.) than an optical element for a 3D image.

FIG. 20 illustrates optical elements, according to an exemplary embodiment. According to the present exemplary embodiment, the optical elements may include a light blocking filter. The light blocking filter is configured to block light and has the same pattern as shown in FIG. 20.

Referring to FIGS. 1 and 20, in an exemplary embodiment, the light blocking filter may be disposed for only one of two eyes of the target object 10 to block light. According to exemplary embodiments, an optical element may not be disposed for the remaining eye, or a light pass filter for passing light may be disposed therefor. According to the present exemplary embodiment, when the MRI apparatus 100a performs an fMRI scan, a light blocking filter may be disposed at a region that corresponds to an eye other than an eye to which a stimulus is to be presented for capturing an fMRI image, i.e., the eye from which the stimulus is to be blocked, and the display 120 may display a predetermined image. For example, to capture an fMRI image of a right eye, a light blocking filter may be disposed in the opening (210a of FIG. 2) of the head coil (130a of FIG. 2) that corresponds to a left eye. In this case, the display 120 may display an image that is intended to present a stimulus to the right eye. The image may include a 2D image and/or a 3D image.

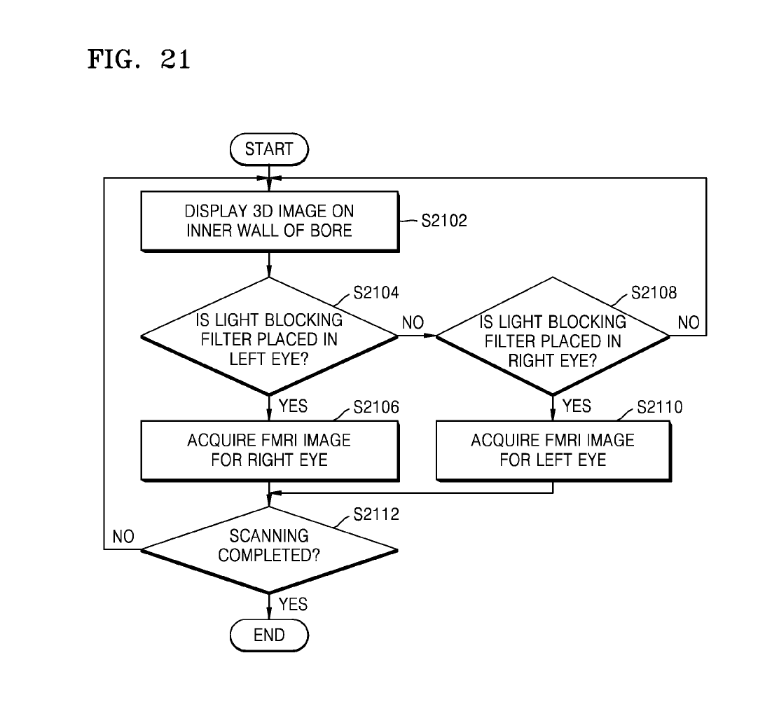

FIG. 21 is a flowchart of a method for controlling the MRI apparatus 100a of FIG. 1, according to an exemplary embodiment.

According to the present embodiment, in operation S2102, the MRI apparatus 100a displays a predetermined image on an inner wall of a bore.

Next, in operation S2104, the MRI apparatus 100a determines whether a light blocking filter is disposed at a region that corresponds to a left eye. According to an exemplary embodiment, the MRI apparatus 100a may determine whether the light blocking filter is disposed at the region corresponding to the left eye based on at least one from among an input received from the target object 10 and an input received from a user. According to another exemplary embodiment, the determination may be performed based on a sensing value from a predetermined sensor in the head coil 130. The sensor may be disposed at a region adjacent to an opening formed in the region corresponding to the left eye, a structure in which an optical element is attachable to and detachable from the head coil 130, etc.

If the light blocking filter is disposed at the region corresponding to the left eye as determined in operation S2104, then in operation S2106, the controller 140 acquires an fMRI image of a right eye.

If the light blocking filter is not disposed at the region corresponding to the left eye as determined in operation S2104, then in operation S2108, the controller 140 determines whether the light blocking filter is disposed at a region that corresponds to a right eye. According to an exemplary embodiment, the controller 140 may determine whether the light blocking filter is disposed at the region corresponding to the right eye based on at least one from among an input received from the target object 10 and an input received from a user. According to another exemplary embodiment, the determination may be performed based on a sensing value from a predetermined sensor in the head coil 130. The sensor may be disposed at a region adjacent to an opening formed in the region corresponding to the right eye, a structure in which an optical element is attachable to and detachable from the head coil 130, etc.

If the light blocking filter is disposed at the region corresponding to the right eye as determined in operation S2108, then in operation S2110, the controller 140 acquires an fMRI image of the left eye. Operations S2102, S2104, S2106, S2108, and S2110 may be repeated until capturing of an fMRI image is completed, as determined in operation S2112.

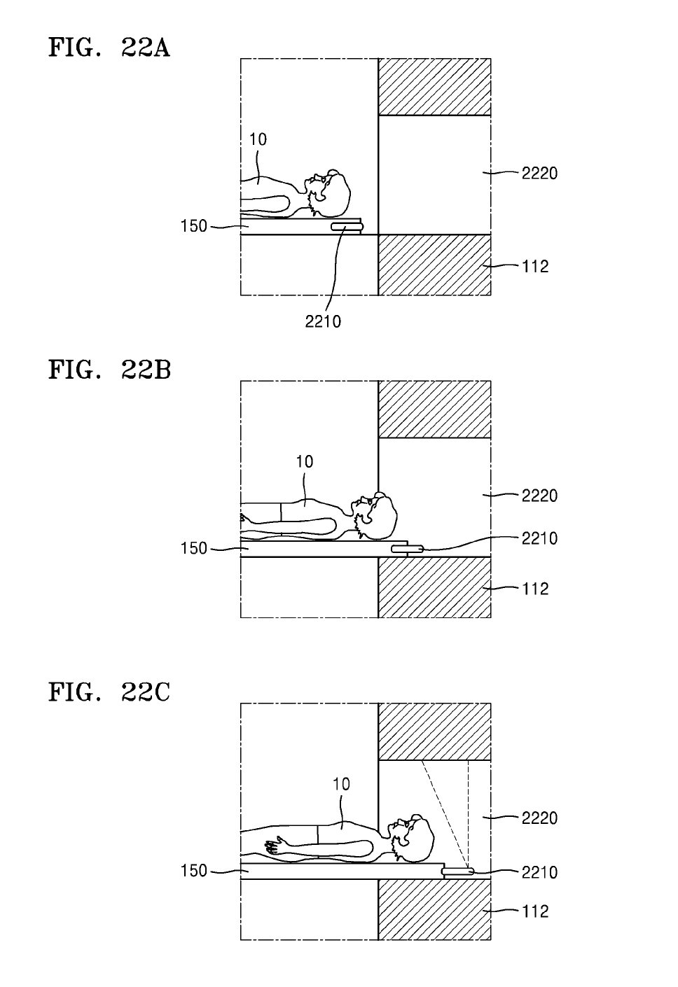

FIGS. 22A, 22B, and 22C illustrate a structure of the display 120 of FIG. 1, according to an exemplary embodiment.

According to the present exemplary embodiment, the display 120 may include an in-bore projector 2210 disposed inside a bore 2220. The in-bore projector 2210 is disposed at a table 150, and within the bore 2220 when the table 150 moves into the bore. In another exemplary embodiment, the in-bore projector 2210 may be fixed inside the bore 2220, e.g., on the inner wall 112 of the bore 2220.

According to an exemplary embodiment, as shown in FIGS. 22A, 22B, and 22C, the in-bore projector 2210 is disposed inside the table 150 to be received in or withdrawn from the table 150. The in-bore projector 2210 may be withdrawn from the table 150 when the table 150 moves from outside the bore 2220 to inside the bore 2220. Furthermore, the in-bore projector 2210 may be received in the table 150 when the table 150 moves from the inside to the outside of the bore 2220. According to the present exemplary embodiment, it is possible to minimize breakage of the in-bore projector 2210 caused by movement of the table 150. In an exemplary embodiment, the in-bore projector 2210 may be received in or withdrawn from the table 150 in accordance with movement of the table 150. Alternatively, the in-bore projector 2210 may be received in or withdrawn from the table 150 by using a power source for moving the table 150.

The in-bore projector 2210 may project an image at a predetermined position on the inner wall 112 of the bore 2220. In an exemplary embodiment, the in-bore projector 2210 is disposed in the table 150, and a position of an image output from the in-bore projector 2210 and displayed on the inner wall 112 of the bore 2220 may vary based on a movement of the table 150. According to the present exemplary embodiment, a position of an image displayed on the inner wall 112 of the bore 2220 may automatically change based on a movement of the table 150, i.e., movement of the target object 10. Thus, according to the present exemplary embodiment, it is possible to adjust a position of an image being displayed so as to correspond to the position of the target object 10 without performing a separate operation for adjusting the position of the image being displayed.

The in-bore projector 2210 may include a circuit configured to minimize the influence of a high magnetic field within the bore 2220. The in-bore projector 2210 may further include an electromagnetic field shield so that it may not affect nor be affected by a high magnetic field and a high electric field within the bore 2220.

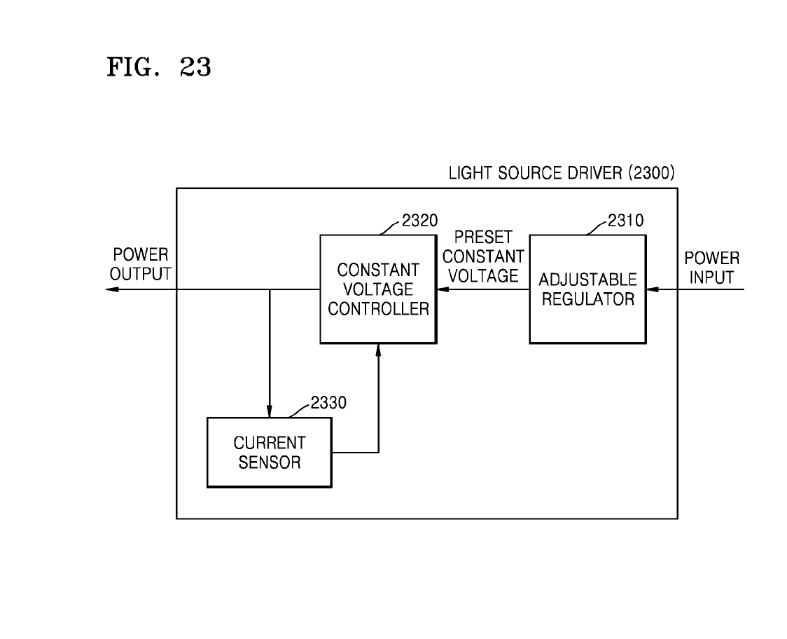

FIG. 23 illustrates a structure of a light source driver 2300 of the in-bore projector 2210 of FIGS. 22A, 22B, and 22C, according to an exemplary embodiment

The light source driver 2300 is required to supply a constant voltage power to a light source of the in-bore projector 2210 even when a rapidly changing current is generated, in order to make the brightness of the light source uniform during an operation of the in-bore projector 2210. The light source driver 2300 may use an adjustable regulator 2310 that does not use an inductor. The adjustable regulator 2310 converts input power into a preset constant voltage and outputs the preset constant voltage. Since the adjustable regulator 2310 does not use an inductor, the adjustable regulator 2310 is not greatly affected by a strong magnetic field within the bore 2220.

However, when only the adjustable regulator 2310 is used, a switching time may be delayed because of its characteristics. Thus, when a current changes rapidly, an output of a constant voltage power may not be maintained at a stable level. Accordingly, the light source driver 2300 may further include a constant voltage controller 2320 and a current sensor 2330. The constant voltage controller 2320 may include a field effect transistor (FET) fast switching device. The current sensor 2330 senses a current supplied to the light source and feeds information that relates to a magnitude of the supplied current to the constant voltage controller 2320. The constant voltage controller 2320 stably supplies a constant voltage power to the light source under fast control of the FET fast switching device, based on the information relating to the magnitude of current sensed by the current sensor 2330, so that the light source may emit a light beam having a uniform brightness for use in the in-bore projector 2210.

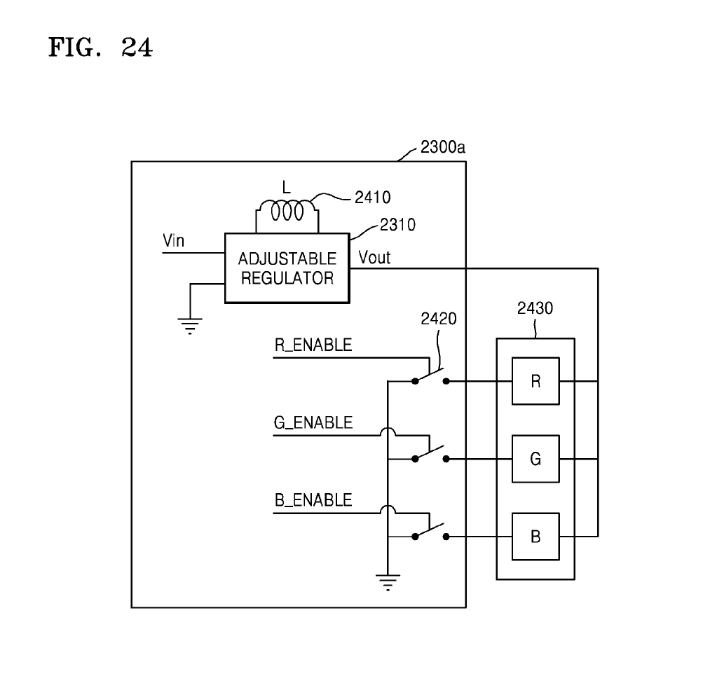

FIG. 24 illustrates a structure of a light source driver 2300a and a light source unit 2430 in the in-bore projector 2210, according to an exemplary embodiment. Voltage conversion by the adjustable regulator 2310 may be performed by adjusting a ratio of on time to off time via a pulse width modulation. Each time that on-off switching occurs during the conversion of voltage, a current flowing through a circuit rapidly changes. Thus, the adjustable regulator 2310 may use a coil type inductor 2410 to adapt to a fast switching operation.

The inductor 2410 may have a cylindrical concentric coil structure in which a wire is wound in the form of a cylinder. The cylinder may have an empty space therein, or may be supported by a non-magnetic material such as Bakelite. The inductor 2410 may not use a magnetic core made of iron or ferrite on which a magnetic force is exerted directly by a magnetic field, and thus, the influence of a strong magnetic field generated within the bore 2220 may be minimized.

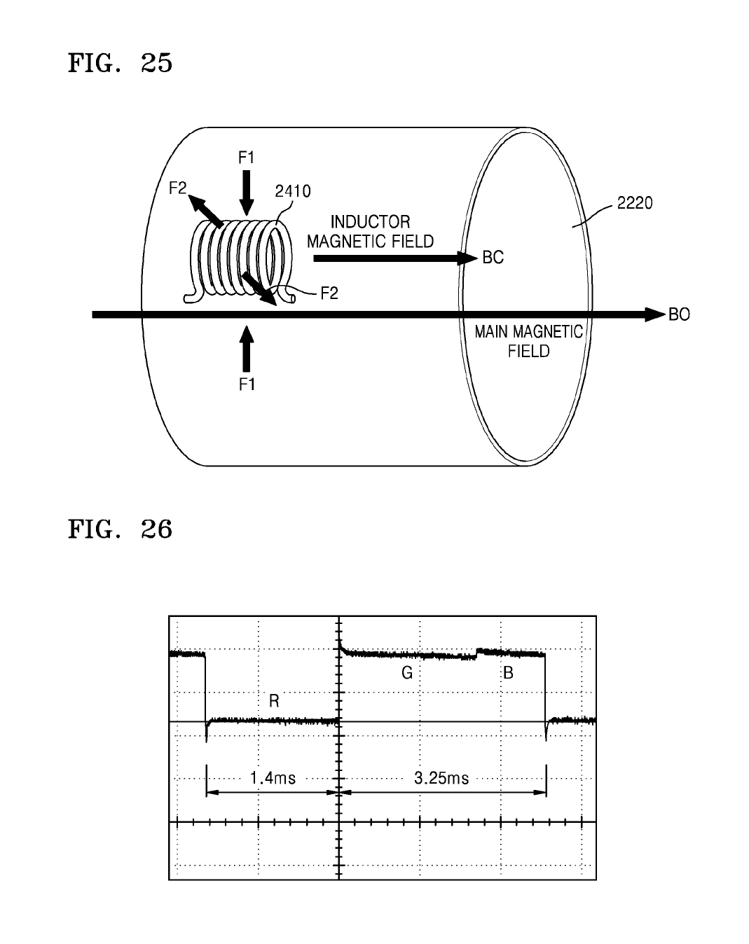

FIG. 25 illustrates the inductor 2410 provided in the adjustable regulator (2310 of FIG. 24) and a bore 2220, according to an exemplary embodiment

According to the present exemplary embodiment, referring to FIG. 25, a central axis of a cylindrical coil that forms the inductor 2410 is horizontal with respect to a direction of a main magnetic field B0 created by a main magnet of the MRI apparatus (100a of FIG. 1). When current is applied to the cylindrical coil, a magnetic field is created within the cylindrical coil in a direction parallel to the central axis of the cylindrical coil. Thus, by placing the central axis of the cylindrical coil horizontal with respect to the direction of the main magnetic field B0 as shown in FIG. 25, the direction of the magnetic field (hereinafter, referred to as an ‘inductor magnetic field’) generated when current flows through the cylindrical coil of the inductor 2410 may be made horizontal with respect to the direction of the main magnetic field B0 created by the main magnet. Since the direction of the main magnetic field B0 may be parallel to the central axis of the cylindrical coil within the bore 2220 as described above, the central axis of the cylindrical coil may be aligned parallel to a central axis of the bore 2220.

An operation of the light source driver 2300a and influence of the main magnetic field B0 created by the main magnet will now be described in detail with reference to FIGS. 24, 25, and 26.

Referring to FIG. 24, the light source driver 2300a supplies a power output from the adjustable regulator 2310 to a red light source, a green light source, and a blue light source in the light source unit 2430. The light source driver 2300a also applies red, green, and blue enable signals R_ENABLE, G_ENABLE, and B_ENABLE, which are generated in response to a light source driving signal, to a switching device 2420 for switching the red, green, and blue light sources in the light source unit 2430.

FIG. 26 illustrates an example of driving signals that are applied to red, green, and blue light sources.

Referring to FIG. 26, a driving signal applied to the red light source is a pulse wave of 1.4 ms having a frequency of 833 Hz, which is within the audible frequency range. Furthermore, driving current applied to the green and blue light sources are pulse waves of 3.25 ms having a frequency of 397.7 Hz, which is within the audible frequency range.

As described above, when the in-bore projector 2210 is located within the bore 2220 during an MRI scan, as shown in FIG. 25, a force may be exerted on the inductor 2410 of the in-bore projector 2210 due to electromagnetic interaction with the main magnetic field B0 created within the bore 2220. In detail, when driving current supplied to the red, green, and blue light sources in the light source unit 2430 flows through a circuit of the light source driver 2300a, a force is exerted on a conductive wire in the coil of the inductor 2410 according to Fleming's left-hand rule. The force acts periodically in an audible frequency range described above.

If the central axis of the cylindrical coil that forms the inductor 2410 is tilted relative to the direction of the main magnetic field B0 created by a main magnet, a force exerted on a conductive wire in the cylindrical coil acts as indicated by an arrow F2 shown in FIG. 25, and thus, a balance of a force is broken. The inductor 2410 in the form of the cylindrical coil creates vibrations, which may be manifested as noise in an audible frequency range. The in-bore projector 2210 may be employed to provide an examinee undergoing MRI scanning with any of various types of content such as a moving image, a picture, scanning state information (e.g., scan time information, scan guide information, and information of a scanned area), and information for use in fMRI, thereby making the examinee feel more at ease. If noise occurs in an audible frequency range, the noise may adversely affect an examination environment for an examinee.

As described above, the MRI apparatus 100a according to the present exemplary embodiment may set a direction in which the inductor 2410 is placed so that a direction of an inductor magnetic field generated when current flows through the inductor 2410 is parallel to a direction of the main magnetic field B0 created by a main magnet, i.e., the central axis of the cylindrical coil that forms the inductor 2410 is horizontal with respect to the main magnetic field B0. By doing so, a force exerted on a conductive wire in the cylindrical coil acts symmetrically, as indicated by an arrow F1 shown in FIG. 25, in order to cancel out vibration. Thus, the MRI apparatus 100a may provide any of various types of content to an examinee without creating noise in the in-bore projector 2210.

The detailed configuration of the light source driver 2300a according to the present exemplary embodiment is described by way of an example and is not limited thereto. In various driving circuits of the related art that also use coils, as described above, occurrence of noise in a coil may be prevented by placing the coil so that a direction of a magnetic field generated when current is applied to the coil is parallel to the direction of a main magnetic field within the bore 2220

Furthermore, although the inductor 2410 in the light source driver 2210 is described as an example of a coil used in the in-bore projector 2210, exemplary embodiments are not limited thereto. Coils may also be used for parts of a circuit block of the in-bore projector 2210 other than the light source driver 2300a. For example, if a coil is used in a power converter that is a part of the circuit block of the in-bore projector 2210, as described above, noise generated in the coil may be suppressed by placing the coil so that a direction of a magnetic field generated by the coil upon application of current is parallel to the direction of the main magnetic field B0 within the bore 2220.

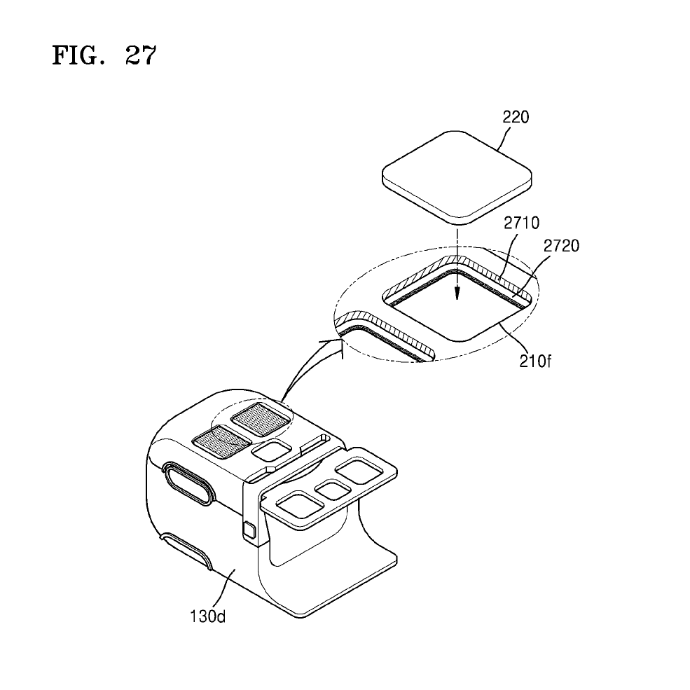

FIG. 27 illustrates a structure in which an optical element 220 is attachable or detachable, according to an exemplary embodiment.

Referring to FIG. 27, the optical element 220 may be attachable to or detachable from a head coil 130d. According to an exemplary embodiment, a head coil 130d may include an optical element holder 2720 that is formed on a circumferential perimeter of an opening 210f and that has a surface step difference 2710 from an outer surface of a frame of the coil 130d. When the optical element holder 2720 is disposed parallel to a horizontal direction of the frame, the optical element 220 may not be inserted via the opening 210f, but may instead be suspended over the optical element holder 2720. For example, the optical element holder 2720 may protrude from the circumferential perimeter of the opening 210f of the head coil 130d.

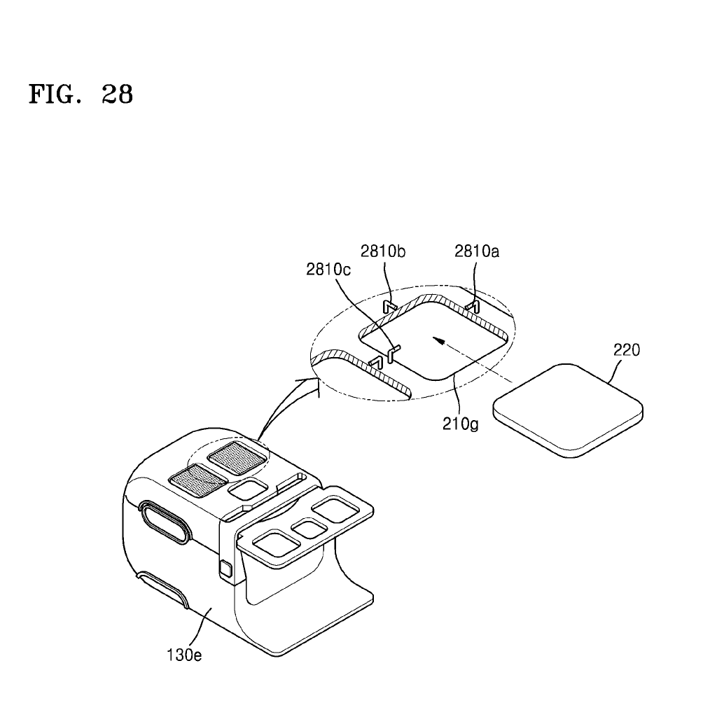

FIG. 28 illustrates a structure in which an optical element 220 is attachable or detachable, according to another exemplary embodiment.

According to an exemplary embodiment, a head coil 130e may include fixing members 2810a, 2810b, and 2810c that are disposed on an outer surface of a frame around an opening 210g and fixed to the optical element 220. The fixing members 2810a, 2810b, and 2810c are constructed to limit movement of the optical element 220 and disposed around the opening 210g. For example, as shown in FIG. 28, the fixing members 2810a, 2810b, and 2810c may have a bent structure.

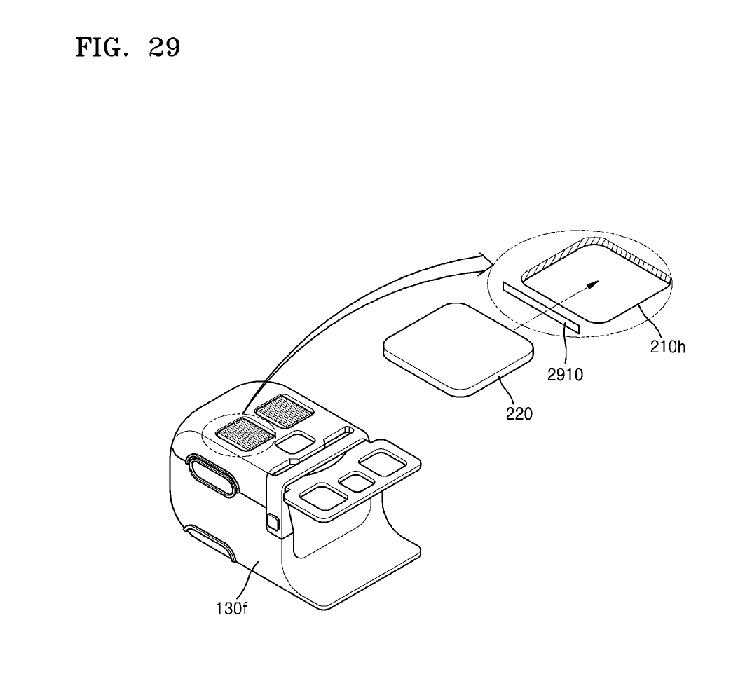

FIG. 29 illustrates a structure in which an optical element 220 is attachable or detachable, according to another exemplary embodiment.

According to an exemplary embodiment, a head coil 130f may include a slot 2910 which is formed in a frame of the head coil 130f and via which the optical element 220 is inserted into an opening 210h of the head coil 130f. The slot 2910 provides a path and a guide along which the optical element 220 passes through the frame into the opening 210h.

According to an exemplary embodiment, the optical element 220 may have a hand grip that facilitates attachment or detachment thereof. For example, the optical element 220 may have a protruding structure that enables a user to hold the optical element 220.

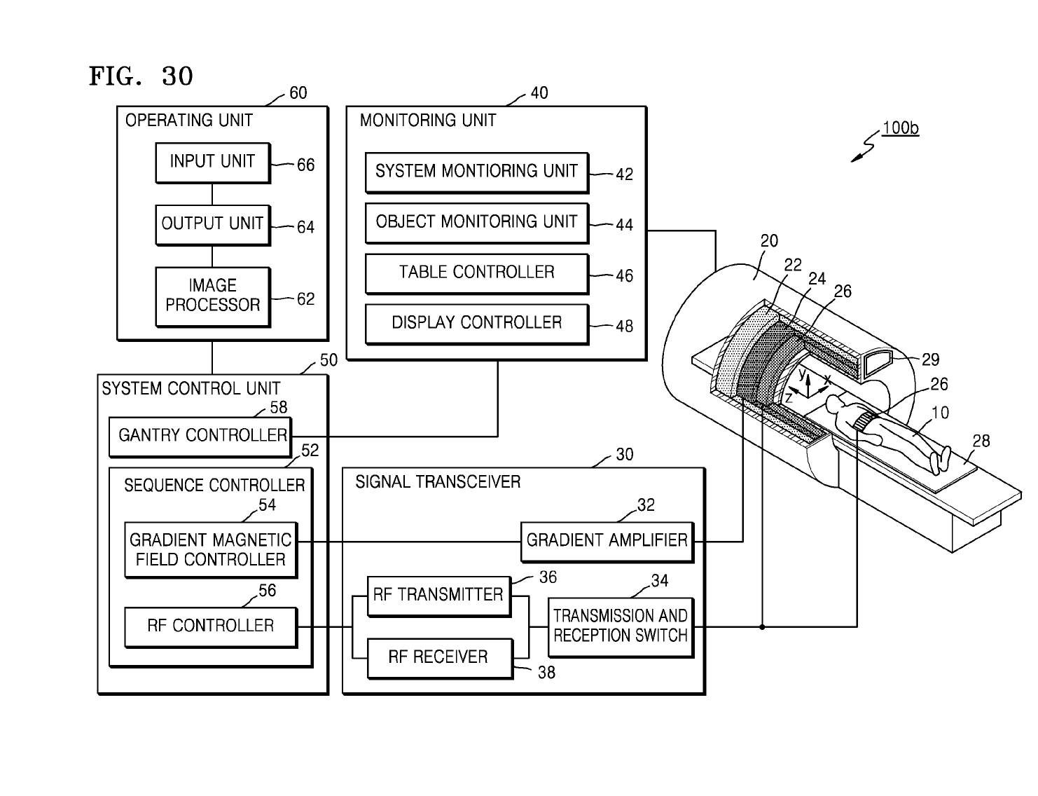

FIG. 30 illustrates a structure of an MRI system 100b, according to an exemplary embodiment. Referring to FIG. 30, the MRI system 100b may include a gantry 20, a signal transceiver 30, a monitoring unit (also referred to herein as a “monitoring device” and/or as a “monitor”) 40, a system control unit (also referred to herein as a “system controller”) 50, and an operating unit (also referred to herein as an “operating device” and/or as an “operator”) 60.

The gantry 20 prevents external emission of electromagnetic waves generated by a main magnet 22, a gradient coil 24, and an RF coil 26. A magnetostatic field and a gradient magnetic field are formed in a bore in the gantry 20, and an RF signal is emitted toward an target object 10.

The main magnet 22, the gradient coil 24, and the RF coil 26 may be arranged in a predetermined direction with respect to the gantry 20. The predetermined direction may be a coaxial cylinder direction with respect to the gantry 20. The target object 10 may be disposed on a table 28 that is capable of being inserted into a cylinder along a horizontal axis of the cylinder.

The main magnet 22 generates a magnetostatic field or a static magnetic field for aligning magnetic dipole moments of atomic nuclei of the target object 10 in a constant direction. A precise and accurate MR image of the target object 10 may be obtained due to a magnetic field generated by the main magnet 22 being strong and uniform.

The gradient coil 24 includes X, Y, and Z coils for generating gradient magnetic fields in X-axis, Y-axis, and Z-axis directions that cross each other at right angles (i.e., directions that are mutually orthogonal to each other). The gradient coil 24 may provide location information relating to each region of the target object 10 by variably inducing resonance frequencies according to the regions of the target object 10.

The RF coil 26 may emit an RF signal toward a patient and receive an MR signal emitted from the patient. In detail, the RF coil 26 may transmit, toward atomic nuclei included in the patient and having precessional motion, an RF signal that has the same frequency as that of the precessional motion, cease the transmission of the RF signal, and then receive an MR signal emitted from the atomic nuclei included in the patient.

For example, in order to cause an atomic nucleus to transition from a low energy state to a high energy state, the RF coil 26 may generate and apply an electromagnetic wave signal that is an RF signal which corresponds to a type of the atomic nucleus, to the target object 10. When the electromagnetic wave signal generated by the RF coil 26 is applied to the atomic nucleus, the atomic nucleus may transit from the low energy state to the high energy state. Then, when electromagnetic waves generated by the RF coil 26 disappear, the atomic nucleus to which the electromagnetic waves were applied transits from the high energy state to the low energy state, thereby emitting electromagnetic waves having a Larmor frequency. In this aspect, when the applying of the electromagnetic wave signal to the atomic nucleus is ceased, an energy level of the atomic nucleus is changed from a high energy level to a low energy level, and thus the atomic nucleus may emit electromagnetic waves having a Larmor frequency. The RF coil 26 may receive electromagnetic wave signals from atomic nuclei included in the target object 10.

The RF coil 26 may be realized as one RF transmitting and receiving coil that has both a function of generating electromagnetic waves each having an RF that corresponds to a type of an atomic nucleus and a function of receiving electromagnetic waves emitted from an atomic nucleus. Alternatively, the RF coil 26 may be realized as a transmission RF coil having a function of generating electromagnetic waves each having an RF that corresponds to a type of an atomic nucleus, and a reception RF coil having a function of receiving electromagnetic waves emitted from an atomic nucleus.

The RF coil 26 may be fixed to the gantry 20 or may be detachable. When the RF coil 26 is detachable, the RF coil 26 may be an RF coil that is configured for a part of the object, such as a head coil, a chest RF coil, a leg RF coil, a neck RF coil, a shoulder RF coil, a wrist RF coil, or an ankle RF coil.

The RF coil 26 may communicate with an external apparatus via wires and/or wirelessly, and may also perform dual tune communication according to a communication frequency band.

The RF coil 26 may communicate with an external apparatus via wires and/or wirelessly, and may also perform dual tune communication according to a communication frequency band.

The RF coil 26 may include any of a transmission exclusive coil, a reception exclusive coil, and/or a transmission and reception coil according to methods of transmitting and receiving an RF signal.

The RF coil 26 may include an RF coil having any of various numbers of channels, such as 16 channels, 32 channels, 72 channels, and 144 channels.

It is hereinafter assumed that the RF coil 26 is an RF multi-coil that includes N coils which correspond to multiple channels, i.e., first through N-th channels. Here, an RF multi-coil may also be referred to as a multi-channel RF coil.

The gantry 20 may further include a display 29 disposed outside the gantry 20 and a display (not shown) disposed inside the gantry 20. The gantry 20 may provide predetermined information to the user or the target object 10 via the display 29 and/or via the display respectively disposed outside and inside the gantry 20.

The signal transceiver 30 may be configured to control the gradient magnetic field formed inside the gantry 20, i.e., in the bore, according to a predetermined MR sequence, and to control a transmission and a reception of an RF signal and an MR signal.

The signal transceiver 30 may include a gradient amplifier 32, a transmission and reception switch 34, an RF data transmitter 36, and RF receiver 38.

The gradient amplifier 32 drives the gradient coil 24 included in the gantry 20, and may supply a pulse signal for generating a gradient magnetic field to the gradient coil 24 under the control of a gradient magnetic field controller 54. By controlling the pulse signal supplied from the gradient amplifier 32 to the gradient coil 24, gradient magnetic fields in X-axis, Y-axis, and Z-axis directions may be synthesized.

The RF transmitter 36 and the RF receiver 38 may be configured to drive the RF coil 26. The RF transmitter 36 may be configured to supply an RF pulse in a Larmor frequency to the RF coil 26, and the RF receiver 38 may be configured to receive an MR signal received by the RF coil 26.

The transmission and reception switch 34 may be configured to adjust transmitting and receiving directions of the RF signal and the MR signal. For example, the transmission and reception switch 34 may be configured to emit the RF signal toward the target object 10 via the RF coil 26 during a transmission mode, and to receive the MR signal from the target object 10 via the RF coil 26 during a reception mode. The transmission and reception switch 34 may be controlled by a control signal output by an RF controller 56.

The monitoring unit 40 may be configured to monitor or control the gantry 20 or devices mounted on the gantry 20. The monitoring unit 40 may include a system monitoring unit (also referred to herein as a “system monitor”) 42, an object monitoring unit (also referred to herein as an “object monitor”) 44, a table controller 46, and a display controller 48.

The system monitoring unit 42 may be configured to monitor and control a state of the magnetostatic field, a state of the gradient magnetic field, a state of the RF signal, a state of the RF coil 26, a state of the table 28, a state of a device measuring body information of the target object 10, a power supply state, a state of a thermal exchanger, and/or a state of a compressor.

The object monitoring unit 44 is configured to monitor a state of the target object 10. In detail, the object monitoring unit 44 may include any one or more of a camera for observing a movement or position of the target object 10, a respiration measurer for measuring the respiration of the target object 10, an electrocardiogram (ECG) measurer for measuring the electrical activity of the target object 10, and/or a temperature measurer for measuring a temperature of the target object 10.

The table controller 46 is configured to control a movement of the table 28 where the target object 10 is positioned. The table controller 46 may control the movement of the table 28 according to a sequence control of a sequence controller 52. For example, during moving imaging of the target object 10, the table controller 46 may continuously or discontinuously move the table 28 according to the sequence control of the sequence controller 52, and thus the target object 10 may be photographed in a field of view (FOV) that is larger than a field of view of the gantry 20.

The display controller 48 is configured to control the display 29 disposed outside the gantry 20 and the display disposed inside the gantry 20. In detail, the display controller 48 may control the display 29 and the display to be powered on or powered off, and may control a screen image to be output on the display 29 and the display. Further, when a speaker is located inside or outside the gantry 20, the display controller 48 may control the speaker to be powered on or powered off, or may control sound to be output via the speaker.

The system control unit 50 may include the sequence controller 52 for controlling a sequence of signals formed in the gantry 20, and a gantry controller 58 for controlling the gantry 20 and the devices mounted on the gantry 20.

The sequence controller 52 may include the gradient magnetic field controller 54 for controlling the gradient amplifier 32, and the RF controller 56 for controlling the RF transmitter 36, the RF receiver 38, and the transmission and reception switch 34. The sequence controller 52 may control the gradient amplifier 32, the RF transmitter 36, the RF receiver 38, and the transmission and reception switch 34 according to a pulse sequence received from the operating unit 60. In this aspect, the pulse sequence includes all information required to control the gradient amplifier 32, the RF transmitter 36, the RF receiver 38, and the transmission and reception switch 34. For example, the pulse sequence may include information that relates to a strength, an application time, and application timing of a pulse signal applied to the gradient coil 24.

The operating unit 60 is configured to request the system control unit 50 to transmit pulse sequence information while controlling overall operations of the MRI system 100b.

The operating unit 60 may include an image processor 62 for receiving and processing the MR signal received by the RF receiver 38, an output unit (also referred to herein as an “output device”) 64, and an input unit (also referred to herein as an “input device”) 66.

The image processor 62 may be configured to process the MR signal received from the RF receiver 38 so as to generate MR image data of the target object 10.

The image processor 62 is configured to perform any one of various signal processes, such as amplification, frequency transformation, phase detection, low frequency amplification, and filtering, on an MR signal received by the RF receiver 38.

The image processor 62 may arrange digital data in a k space of a memory, and rearrange the digital data into image data via 2D and/or 3D Fourier transformation.

The image processor 62 may perform a composition process or difference calculation process on image data if required. The composition process may include an addition process on a pixel and/or a maximum intensity projection (MIP) process. The image processor 62 may store not only the rearranged image data, but also image data upon which a composition process or a difference calculation process is performed, in a memory (not shown) or an external server.

The image processor 62 may perform any of the signal processes on the MR signal in parallel. For example, the image processor 62 may perform a signal process on a plurality of MR signals received by a multi-channel RF coil in parallel so as to rearrange the plurality of MR signals into image data.

The output unit 64 may be configured to output image data generated or rearranged by the image processor 62 to the user. The output unit 64 may also output information required for the user to manipulate the MRI system 100b such as a user interface (UI), user information, or object information. Examples of an output unit may include a speaker, a printer, a cathode ray tube (CRT) display, a liquid crystal display (LCD), a plasma display panel (PDP), an organic light emitting diode (OLED) display, a field emission display (FED), a light emitting diode (LED) display, a vacuum fluorescent display (VFD), a digital light processing (DLP) display, a flat panel display (FPD), a three-dimensional (3D) display, a transparent display, and/or any of other various output devices well known to one of ordinary skill in the art.

The user may input object information, parameter information, a scan condition, a pulse sequence, or information about image composition or difference calculation by using an input unit 66. An input unit 66 may include any one or more of a keyboard, a mouse, a track ball, a voice recognizer, a gesture recognizer, a touch pad, and/or any one of other various input devices that are well known to one of ordinary skill in the art.

The signal transceiver 30, the monitoring unit 40, the system control unit 50, and the operating unit 60 are separate components in FIG. 30, but it will be apparent to one of ordinary skill in the art that respective functions of the signal transceiver 30, the monitoring unit 40, the system control unit 50, and the operating unit 60 may be performed by another component. For example, the image processor 62 converts the MR signal received from the RF receiver 38 into a digital signal in FIG. 1, but alternatively, the conversion of the MR signal into the digital signal may be performed by RF receiver 38 or the RF coil 26.

The gantry 20, the RF coil 26, the signal transceiver 30, the monitoring unit 40, the system control unit 50, and the operating unit 60 may be connected to each other by wire or wirelessly, and when they are connected wirelessly, the MRI system may further include an apparatus (not shown) for synchronizing clock signals therebetween. Communication between the gantry 20, the RF coil 26, the signal transceiver 30, the monitoring unit 40, the system control unit 50, and the operating unit 60 may be performed by using a high-speed digital interface, such as low voltage differential signaling (LVDS), asynchronous serial communication, such as a universal asynchronous receiver transmitter (UART), a low-delay network protocol, such as error synchronous serial communication or a controller area network (CAN), optical communication, or any of other various communication methods that are well known to one of ordinary skill in the art.

The signal transceiver 30, the monitoring unit 40, the system control unit 50, and the operating unit 60 may correspond to the controller 140 shown in FIG. 1. The table 28 may correspond to the table 150 shown in FIG. 1.

While one or more exemplary embodiments have been described with reference to the figures, it will be understood by those of ordinary skill in the art that various changes in form and details may be made therein without departing from the spirit and scope as defined by the following claims. Accordingly, the above exemplary embodiments and all aspects thereof are examples only and are not limiting.

What is claimed is:

1. A magnetic resonance imaging (MRI) apparatus comprising:
a display configured to display a three-dimensional (3D) image on an inner wall of a bore within a gantry;
a head coil comprising at least one opening formed at a region that corresponds to eyes of a target object and an optical element disposed in the at least one opening; and
a controller configured to adjust a perspective of the 3D image based on at least one from among an input received from the target object and an input received from a user.

2. The MRI apparatus of claim 1, wherein the 3D image comprises a left-eye image polarized in a first direction and a right-eye image polarized in a second direction, and
wherein the optical element comprises a left-eye polarizing filter configured to polarize light in the first direction and a right-eye polarizing filter configured to polarize light in the second direction.

3. The MRI apparatus of claim 1, wherein the 3D image is generated by combining a left-eye image represented by a first color component with a right-eye image represented by a second color component, and
wherein the optical element comprises a left-eye color filter configured to pass the first color component and a right-eye color filter configured to pass the second color component.

4. The MRI apparatus of claim 1, wherein the optical element is further configured to facilitate a reception of light by a first one of the eyes of the target object and to block light such that a second one of the eyes of the target object does not receive the blocked light, and
wherein the controller is further configured to acquire a functional MRI (fMRI) image.

5. The MRI apparatus of claim 1, wherein the head coil further comprises at least one vision correction lens disposed in the at least one opening.

6. The MRI apparatus of claim 1, wherein the 3D image comprises a background image and a content image, and
wherein the controller is further configured to change a perspective of the background image based on the at least one from among the input received from the target object and the input received from the user.

7. The MRI apparatus of claim 1, wherein the 3D image comprises at least one object that is focused behind the inner wall of the bore with respect to the target object.

8. The MRI apparatus of claim 1, wherein the display comprises at least one projector configured to project the 3D image onto the inner wall of the bore.

9. The MRI apparatus of claim 8, wherein the at least one projector is disposed inside the bore.

10. The MRI apparatus of claim 8, further comprising a table on which the target object is placed and which is configured to enter and to exit the bore, and
wherein the at least one projector is attached to the table and is disposed within the bore when the table is inside the bore.

11. The MRI apparatus of claim 1, wherein the inner wall of the bore has a printed background pattern, and
wherein the 3D image comprises at least one object that is focused in front of the inner wall of the bore with respect to the target object.

12. The MRI apparatus of claim 1, wherein the optical element is attachable to the at least one opening and detachable from the at least one opening.

13. The MRI apparatus of claim 1, wherein the optical element includes at least one from among a filter for viewing a 3D image, a light blocking filter, and a vision correction lens.

14. The MRI apparatus of claim 12, wherein the head coil further comprises a frame which comprises an optical element holder that is formed around the at least one opening to have a surface step difference from an outer surface of the frame.

15. The MRI apparatus of claim 14, wherein the frame of the head coil further comprises a slot for providing a guide via which the optical element is inserted into the at least one opening.

16. The MRI apparatus of claim 1, further comprising a table on which the target object is placed and which is configured to enter and to exit the bore, and
wherein the display is further configured to change a position at which the 3D image is displayed on the inner wall of the bore based on a distance traversed by the table while entering the bore.

17. The MRI apparatus of claim 1, wherein the controller is further configured to correct a distortion in the 3D image displayed on the inner wall of the bore.

18. A method for controlling a magnetic resonance imaging (MRI) apparatus, the method comprising:
displaying a three-dimensional (3D) image on an inner wall of a bore within a gantry of the MRI apparatus; and
adjusting a perspective of the 3D image based on at least one from among an input received from a target object and an input received from a user,
wherein the 3D image comprises a left-eye image and a right-eye image,
wherein the left-eye image has at least one optical characteristic that corresponds to a left-eye optical filter disposed in an opening formed at a region in a head coil of the MRI apparatus that corresponds to a left eye of the target object, and
wherein the right-eye image has at least one optical characteristic that corresponds to a right-eye optical filter disposed in an opening formed at a region in the head coil of the MRI apparatus that corresponds to a right eye of the target object.

19. The method of claim 18, wherein the 3D image comprises a left-eye image polarized in a first direction and a right-eye image polarized in a second direction, and
wherein the left-eye optical filter includes a left-eye polarizing filter configured to polarize light in the first direction, and the right-eye optical filter includes a right-eye polarizing filter configured to polarize light in the second direction.

20. The method of claim 18, wherein the 3D image is generated by combining a left-eye image represented by a first color component with a right-eye image represented by a second color component, and
wherein the left-eye optical filter includes a left-eye color filter configured to pass the first color component, and the right-eye optical filter includes a right-eye color filter configured to pass the second color component.

21. The method of claim 18, wherein a first one of the left-eye optical filter and the right-eye optical filter is configured to facilitate a reception of light by a corresponding one of the left eye of the target object and the right eye of the target object, and a second one of the left-eye optical filter and the right-eye optical filter is configured to block light such that a corresponding one of the left eye of the target object and the right eye of the target object does not receive the blocked light, and
the method further comprises acquiring a functional MRI (fMRI) image.

22. The method of claim 18, wherein the 3D image comprises a background image and a content image, and
the method further comprises changing a perspective of the background image based on the at least one from among the input received from the target object and the input received from the user.

23. The method of claim 18, wherein the 3D image comprises at least one object that is focused behind the inner wall of the bore with respect to the target object.

24. The method of claim 18, further comprising projecting the 3D image onto the inner wall of the bore by using at least one projector.

25. The method of claim 18, further comprising changing a position where the 3D image is displayed on the inner wall of the bore based on a distance traversed by a table while entering the bore.

26. The method of claim 18, further comprising correcting a distortion in the 3D image displayed on the inner wall of the bore.

27. A magnetic resonance imaging (MRI) apparatus comprising:
a projector configured to project a composite image onto an inner wall of a bore within a gantry, the composite image including a left-eye image and a right-eye image;
a headgear comprising at least one opening formed at a region that corresponds to eyes of a target object and an optical element disposed in the at least one opening; and
a controller configured to adjust a perspective of the composite image based on at least one from among an input received from the target object and an input received from a user.

28. The MRI apparatus of claim 27, wherein the left-eye image is polarized in a first direction and the right-eye image is polarized in a second direction, and
wherein the optical element comprises a left-eye polarizing filter configured to polarize light in the first direction and a right-eye polarizing filter configured to polarize light in the second direction.

29. The MRI apparatus of claim 27, wherein the left-eye image corresponds to a first color component and the right-eye image corresponds to a second color component, and
wherein the optical element comprises a left-eye color filter configured to pass the first color component and a right-eye color filter configured to pass the second color component.

30. The MRI apparatus of claim 27, wherein the optical element is further configured to facilitate a reception of light by a left eye of the target object and to block light such that a right eye of the target object does not receive the blocked light, and
wherein the controller is further configured to acquire a functional MRI (fMRI) image.

31. The MRI apparatus of claim 27, wherein the optical element is further configured to facilitate a reception of light by a right eye of the target object and to block light such that a left eye of the target object does not receive the blocked light, and
wherein the controller is further configured to acquire a functional MRI (fMRI) image.
